(19) United States
(12) Patent Application Publication
(10) Pub. No.: US 2002/0010184 A1
(43) Pub. Date: Jan. 24, 2002

(54) Inhibitors of prenyl-protein transferase

(76) Inventors: Christopher J. Dinsmore, Schwenksville, PA (US); Jeffrey M. Bergman, Perkasie, PA (US)

Correspondence Address: MERCK AND CO INC, P O BOX 2000, RAHWAY, NJ, 070650907

(21) Appl. No.: 09/770,967

(22) Filed: Jan. 26, 2001

Related U.S. Application Data
(60) Provisional application No. 60/183,651, filed on Feb. 18, 2000.

(51) Int. Cl. A61K 31/496; C07D 403/00

(52) U.S. Cl. 514/253.09; 514/254.05; 544/360; 544/370

(57) ABSTRACT

The present invention comprises piperazinone-containing compounds which inhibit prenyl-protein transferases, including famesyl-protein transferase and geranylgeranyl-protein transferase type I. Such therapeutic compounds are useful in the treatment of cancer.

### BACKGROUND OF THE INVENTION

The Ras proteins (Ha-Ras, Ki4a-Ras, Ki4b-Ras and N-Ras) are part of a signalling pathway that links cell surface growth factor receptors to nuclear signals initiating cellular proliferation. Biological and biochemical studies of Ras action indicate that Ras functions like a G-regulatory protein. In the inactive state, Ras is bound to GDP. Upon growth factor receptor activation Ras is induced to exchange GDP for GTP and undergoes a conformational change. The GTP-bound form of Ras propagates the growth stimulatory signal until the signal is terminated by the intrinsic GTPase activity of Ras, which returns the protein to its inactive GDP bound form (D. R. Lowy and D. M. Willumsen, Ann. Rev. Biochem. 62:851-891 (1993)). Mutated ras genes (Ha-ras, Ki4a-ras, Ki4b-ras and N-ras) are found in many human cancers, including colorectal carcinoma, exocrine pancreatic carcinoma, and myeloid leukemias. The protein products of these genes are defective in their GTPase activity and constitutively transmit a growth stimulatory signal.

Ras must be localized to the plasma membrane for both normal and oncogenic functions. At least 3 post-translational modifications are involved with Ras membrane localization, and all 3 modifications occur at the C-terminus of Ras. The Ras C-terminus contains a sequence motif termed a “CAAX” or “Cys-Aaa¹-Aaa²-Xaa” box (Cys is cysteine, Aaa is an aliphatic amino acid, the Xaa is any amino acid) (Willumsen et al., Nature 310:583-586 (1984)). Depending on the specific sequence, this motif serves as a signal sequence for the enzymes farnesyl-protein transferase or geranylgeranyl-protein transferase, which catalyze the alkylation of the cysteine residue of the CAAX motif with a C₁₅ or C₂₀ isoprenoid, respectively. Such enzymes may be generally termed prenyl-protein transferases. (S. Clarke., Ann. Rev. Biochem. 61:355-386 (1992); W.R. Schafer and J. Rine, Ann. Rev. Genetics 30:209-237 (1992)). The Ras protein is one of several proteins that are known to undergo post-translational farnesylation. Other farnesylated proteins include the Ras-related GTP-binding proteins such as Rho, fungal mating factors, the nuclear lamins, and the gamma subunit of transducin. James, et al., J. Biol. Chem. 269, 14182 (1994) have identified a peroxisome associated protein Pxf which is also famesylated. James, et al., have also suggested that there are farnesylated proteins of unknown structure and function in addition to those listed above.

The Ras protein is one of several proteins that are known to undergo post-translational modification. Farnesyl-protein transferase utilizes farnesyl pyrophosphate to covalently modify the Cys thiol group of the Ras CAAX box with a farnesyl group (Reiss et al., Cell, 62:81-88 (1990); Schaber et al., J. Biol. Chem., 265:14701-14704 (1990); Schaferet al., Science, 249:1133-1139 (1990); Manne et al., Proc. Natl. Acad. Sci USA, 87:7541-7545 (1990)).

Mammalian cells express four types of Ras proteins (H-, N-, K4A-, and K4B-Ras) among which K4B-Ras is the most frequently mutated form of Ras in human cancers. The genes that encode these proteins are abbreviated H-ras, N-ras, K4A-ras and K4B-ras respectively. H-ras is an abbreviation for Harvey-ras. K4A-ras and K4B-ras are abbreviations for the Kirsten splice variants of ras that contain the 4A and 4B exons, respectively. Inhibition of farnesyl-protein transferase has been shown to block the growth of H-ras-transformed cells in soft agar and to modify other aspects of their transformed phenotype. It has also been demonstrated that certain inhibitors of farnesyl-protein transferase selectively block the processing of the H-Ras oncoprotein intracellularly (N. E. Kohl et al., Science, 260:1934-1937 (1993) and G. L. James et al., Science, 260:1937-1942 (1993). Recently, it has been shown that an inhibitor of farnesyl-protein transferase blocks the growth of H-ras-dependent tumors in nude mice (N. E. Kohl et al., Proc. Natl. Acad. Sci U.S.A., 91:9141-9145 (1994) and induces regression of mammary and salivary carcinomas in H-ras transgenic mice (N. E. Kohl et al., Nature Medicine, 1:792-797 (1995).

Mutated ras genes are found in many human cancers, including colorectal carcinoma, exocrine pancreatic carcinoma, and myeloid leukemias. The protein products of these genes are defective in their GTPase activity and constitutively transmit a growth stimulatory signal.

Prenylation of proteins by prenyl-protein transferases represents a class of post-translational modification (Glomset, J. A., Gelb, M. H., and Farnsworth, C. C. (1990). Trends Biochem. Sci. 15, 139-142; Maltese, W. A. (1990). FASEB J. 4, 3319-3328). This modification typically is required for the membrane localization and function of these proteins. Prenylated proteins share characteristic C-terminal sequences including CAAX (C, Cys; A, an aliphatic amino acid; X, another amino acid), XXCC, or XCXC. Three post-translational processing steps have been described for proteins having a C-terminal CAAX sequence: addition of either a 15 carbon (farnesyl) or 20 carbon (geranylgeranyl) isoprenoid to the Cys residue, proteolytic cleavage of the last 3 amino acids, and methylation of the new C-terminal carboxylate (Cox, A. D. and Der, C. J. (1992a). Critical Rev. Oncogenesis 3:365-400; Newman, C. M. H. and Magee, A. I. (1993). Biochim. Biophys. Acta 1155:79-96). Some proteins may also have a fourth modification: palmitoylation of one or two Cys residues N-terminal to the farnesylated Cys. While some mammalian cell proteins terminating in XCXC are carboxymethylated, it is not clear whether carboxy methylation follows prenylation of proteins terminating with a XXCC motif (Clarke, S. (1992). Annu. Rev. Biochem. 61, 355-386). For all of the prenylated proteins, addition of the isoprenoid is the first step and is required for the subsequent steps (Cox, A. D. and Der, C. J. (1992a). Critical Rev. Oncogenesis 3:365-400; Cox, A. D. and Der, C. J. (1992b) Current Opinion Cell Biol. 4:1008-1016).

The prenylation reactions have been shown genetically to be essential for the function of a variety of proteins (Clarke, 1992; Cox and Der, 1992a; Gibbs, J. B. (1991). Cell 65: 1-4; Newman and Magee, 1993; Schafer and Rine, 1992). This requirement often is demonstrated by mutating the CaaX Cys acceptors so that the proteins can no longer be prenylated. The resulting proteins are devoid of their central biological activity. These studies provide a genetic “proof of principle” indicating that inhibitors of prenylation can alter the physiological responses regulated by prenylated proteins.

Three enzymes have been described that catalyze protein prenylation: farnesyl-protein transferase (FPTase), geranylgeranyl-protein transferase type I (GGPTase-I), and geranylgeranyl-protein transferase type-II (GGPTase-II, also called Rab GGPTase). These enzymes are found in both yeast and mammalian cells (Clarke, 1992; Schafer, W. R. and Rine, J. (1992) Annu. Rev. Genet. 30:209-237). Each of these enzymes selectively uses famesyl diphosphate or geranyl-geranyl diphosphate as the isoprenoid donor and selectively recognizes the protein substrate. FPTase farnesylates CaaX-containing proteins that end with Ser, Met, Cys, Gln or Ala. For FPTase, CaaX tetrapeptides comprise the minimum region required for interaction of the protein substrate with the enzyme. The enzymological characterization of these three enzymes has demonstrated that it is possible to selectively inhibit one with little inhibitory effect on the others (Moores, S. L., Schaber, M. D., Mosser, S. D., Rands, E., OHara, M. B., Garsky, V. M., Marshall, M. S., Pompliano, D. L., and Gibbs, J. B., J. Biol. Chem., 266:17438 (1991), U.S. Pat. No. 5,470,832).

Inhibition of farnesyl-protein transferase has been shown to block the growth of Ras-transformed cells in soft agar and to modify other aspects of their transformed phenotype. It has also been demonstrated that certain inhibitors of farnesyl-protein transferase selectively block the processing of the Ras oncoprotein intracellularly (N. E. Kohl et al., Science, 260:1934-1937 (1993) and G. L. James et al., Science, 260:1937-1942 (1993). Recently, it has been shown that an inhibitor of farnesyl-protein transferase blocks the growth of ras-dependent tumors in nude mice (N. E. Kohl et al., Proc. Natl. Acad. Sci U.S.A., 91:9141-9145 (1994) and induces regression of mammary and salivary carcinomas in ras transgenic mice (N. E. Kohl et al., Nature Medicine, 1:792-797 (1995).

Indirect inhibition of farnesyl-protein transferase in vivo has been demonstrated with lovastatin (Merck & Co., Rahway, N.J.) and compactin (Hancock et al., ibid; Casey et al., ibid; Schafer et al., Science 245:379 (1989)). These drugs inhibit HMG-CoA reductase, the rate limiting enzyme for the production of poly-isoprenoids including farnesyl pyrophosphate. Farnesyl-protein transferase utilizes farnesyl pyrophosphate to covalently modify the Cys thiol group of the Ras CAAX box with a farnesyl group (Reiss et al., Cell, 62:81-88 (1990); Schaber et al., J. Biol. Chem., 265:14701-14704 (1990); Schafer et al., Science, 249:1133-1139 (1990); Manne et al., Proc. Natl. Acad. Sci USA, 87:7541-7545 (1990)). Inhibition of farnesyl pyrophosphate biosynthesis by inhibiting HMG-CoA reductase blocks Ras membrane localization in cultured cells. However, direct inhibition of farnesyl-protein transferase would be more specific and attended by fewer side effects than would occur with the required dose of a general inhibitor of isoprene biosynthesis.

Inhibitors of farnesyl-protein transferase (FPTase) have been described in two general classes. The first are analogs of farnesyl diphosphate (FPP), while the second class of inhibitors is related to the protein substrates (e.g., Ras) for the enzyme. The peptide derived inhibitors that have been described are generally cysteine containing molecules that are related to the CAAX motif that is the signal for protein prenylation. (Schaber et al., ibid; Reiss et. al., ibid; Reiss et al., PNAS, 88:732-736 (1991)). Such inhibitors may inhibit protein prenylation while serving as alternate substrates for the farnesyl-protein transferase enzyme, or may be purely competitive inhibitors (U.S. Pat. No. 5,141,851, University of Texas; N. E. Kohl et al., Science, 260:1934-1937 (1993); Graham, et al., J. Med. Chem., 37, 725 (1994)). In general, deletion of the thiol from a CAAX derivative has been shown to dramatically reduce the inhibitory potency of the compound. However, the thiol group potentially places limitations on the therapeutic application of FPTase inhibitors with respect to pharmacokinetics, pharmacodynamics and toxicity. Therefore, a functional replacement for the thiol is desirable.

It has been disclosed that the lysine-rich region and terminal CVIM sequence of the C-terminus of K-RasB confer resistance to inhibition of the cellular processing of that protein by certain selective FPTase inhibitors. (James, et al., J. Biol. Chem. 270, 6221 (1995) Those FPTase inhibitors were effective in inhibiting the processing of H-Ras proteins. James et al., suggested that prenylation of the K4B-Ras protein by GGTase-I contributed to the resistance to the selective FPTase inhibitors.

It has recently been reported that farnesyl-protein transferase inhibitors are inhibitors of proliferation of vascular smooth muscle cells and are therefore useful in the prevention and therapy of arteriosclerosis and diabetic disturbance of blood vessels (JP H7-112930).

It has recently been disclosed that certain tricyclic compounds which optionally incorporate a piperidine moiety are inhibitors of FPTase (WO 95/10514, WO 95/10515 and WO 95/10516). Imidazole-containing inhibitors of farnesyl protein transferase have also been disclosed (WO 95/09001 and EP 0 675 112 A1). It has also been disclosed that certain compounds which incorporate a pyrrolidine moiety are inhibitors of FPTase (WO 97/37900, and U.S. Pat. Nos. 5,627,202 and 5,661,161).

It is, therefore, an object of this invention to develop compounds that will inhibit prenyl-protein transferase and thus, the post-translational isoprenylation of proteins. It is a further object of this invention to develop chemotherapeutic compositions containing the compounds of this invention and methods for producing the compounds of this invention.

### SUMMARY OF THE INVENTION

The present invention comprises piperazinone-containing compounds which inhibit prenyl-protein transferases. Further contained in this invention are chemotherapeutic compositions containing these prenyl-protein transferase inhibitors and methods for their production.

The compounds of this invention are illustrated by the formula A:

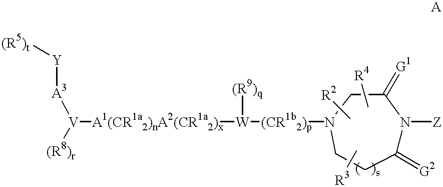

### DETAILED DESCRIPTION OF THE INVENTION

The compounds of this invention are useful in the inhibition of prenyl-protein transferases and the prenylation of the oncogene protein Ras. In a first embodiment of this invention, the inhibitors of prenyl-protein transferases are illustrated by formula A:

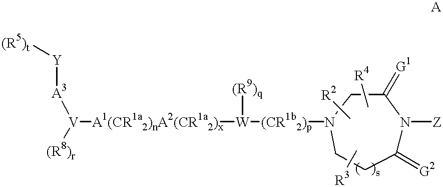

wherein:

R^{1a} and R^{1b} are independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, R¹⁰O—, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, or

c) unsubstituted or substituted C₁-C₆ alkyl wherein the substitutent on the substituted C₁-C₆ alkyl is selected from unsubstituted or substituted aryl, unsubstituted or substituted heterocycle, unsubstituted or substituted C₃-C₁₀ cycloalkyl, C₂-C₈ alkenyl, C₂-C₈ alkynyl, R¹⁰O—, R¹¹S(O)ₘ—, R¹⁰C(O)NR¹⁰—, (R⁰)₂NC(O)—, (R¹⁰)₂NC(O)NR¹⁰—, CN, R¹⁰OC(O)—, R¹⁰OC(O)—, —N(R¹⁰)₂, and R¹¹OC(O)NR¹⁰—;

R² and R³ are independently selected from: H, unsubstituted or substituted C₁₋₆ alkyl, unsubstituted or substituted C₂₋₈ alkenyl, unsubstituted or substituted C₂₋₈ alkynyl, unsubstituted or substituted aryl, unsubstituted or substituted heterocycle,

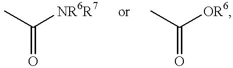

wherein the substituted group is substituted with one or more of:

1) aryl or heterocycle, unsubstituted or substituted with:

a) C₁₋₆ alkyl,

b) (CH₂)pOR⁶,

c) (CH₂)pNR⁶R⁷,

d) halogen,

e) CN,

2) C₃₋₆ cycloalkyl,

3) OR⁶,

4) SR^{6a}, S(O)R^{6a}, SO₂R^{6a},

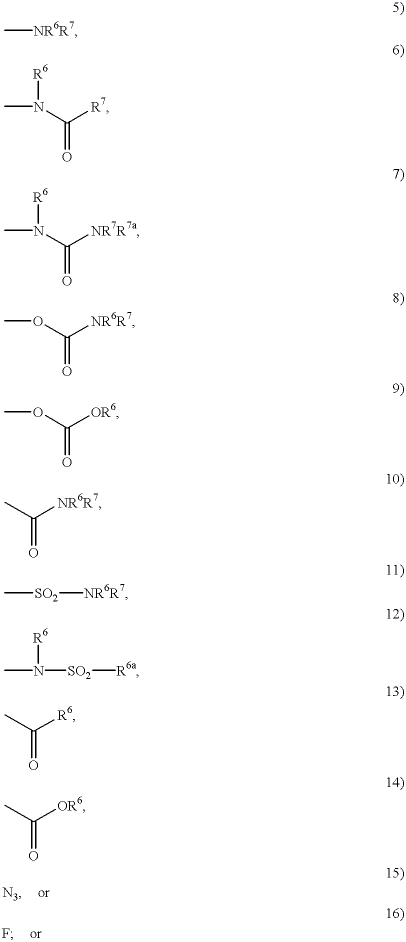

R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;

R⁴ is selected from H and unsubstituted or substituted C₁-C₆ alkyl;

and any two of R², R³ or R⁴ are optionally attached to the same carbon atom;

R⁵ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰OC(O)NR¹⁰—, (R¹⁰)₂NC(O)—, (R¹⁰)₂NC(O)NR¹⁰—, CN, NO₂, R¹⁰OC(O)—, R¹⁰OC(O)—, —N(R¹⁰)₂, or R¹¹OC(O)NR¹⁰—, and

c) C₁-C₆ alkyl, unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;

R⁶, R⁷ and R^{7a} are independently selected from: H, C₁-C₆ alkyl, C₃₋₆ cycloalkyl, heterocycle, aryl, aroyl, heteroaroyl, arylsulfonyl, heteroarylsulfonyl, unsubstituted or substituted with:

a) C₁₋₆ alkoxy,

b) C₁-C₂₀ alkyl

c) aryl or heterocycle,

d) halogen,

e) HO,

f) —C(O)R¹¹,

g) —SO₂R¹¹, or

h) N(R¹⁰)₂; or

R⁶ and R⁷ may be joined in a ring;

R⁷ and R⁷a may be joined in a ring;

R⁶a is selected from: C₁-C₆ alkyl, C₃₋₆ cycloalkyl, heterocycle, aryl, aroyl, heteroaroyl, arylsulfonyl, heteroarylsulfonyl, unsubstituted or substituted with:

a) C₁₋₄ alkoxy,

b) C₁-C₂₀ alkyl

c) aryl or heterocycle,

d) halogen,

e) HO,

f) —C(O)R¹¹,

g) —SO₂R¹¹, or

h) N(R¹⁰)_{2;}

R⁸ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹C(O)NR¹⁰—;

R⁹ is selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, heterocycle, C₃-C₁₀ cycloalkyl, perfluoroalkyl, halo, R¹⁰O—, R¹¹ S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;

R¹⁰ is independently selected from hydrogen, unsubstituted or substituted C₁-C₆ alkyl, perfluoroalkyl, unsubstituted or substituted aralkyl, and unsubstituted or substituted aryl; R¹¹ is independently selected from unsubstituted or substituted C₁-C₆ alkyl and unsubstituted or substituted aryl;

A¹ and A² are independently selected from: a bond, —CH═CH—, —C≡C—, —C(O)—, —C(O)NR¹⁰—, —NR¹⁰C(O)—, O, —N(R¹⁰)—, —S(O)₂N(R¹⁰)—, —N(R¹⁰)S(O)₂—, or S(O)ₘ;

A³ is selected from —C(O)—, —C(R^{1a})₂—, O, —N(R¹⁰)— and S(O)ₘ;

G¹ or G² is selected from H₂ or O, provided that if G¹ is O then G² is H₂ and if G² is O, then G¹ is H₂;

V is selected from:

a) heterocycle, and

b) aryl,

W is a heterocycle;

Y is heteroaryl;

Z is a unsubstituted or substituted group selected from aryl, heteroaryl, arylmethyl, heteroarylmethyl, arylsulfonyl, heteroarylsulfonyl, wherein the substituted group is substituted with one or more of the following:

1) C₁-C₆ alkyl, unsubstituted or substituted with:

a) C₁₋₆ alkoxy,

b) NR⁶R⁷,

c) C₃₋₆ cycloalkyl,

d) aryl or heterocycle,

e) HO,

f) —S(O)ₘR^{6a}, or

g) —C(O)NR⁶R⁷,

2) unsubstituted or substituted aryl or unsubstituted or substituted heterocycle,

3) halogen,

4) OR⁶,

5) NR⁶R⁷,

6) CN,

7) NO₂,

8) CF₃,

9) —S(O)ₘR^{6a},

10) —C(O)NR⁶R⁷,

11) —OCF₃,

12) unsubstituted or substituted C₁₋₆ alkoxy,

13) C₂-C₈ alkenyl,

14) C₂-C₈ alkynyl, or

15) C₃-C₁₀ cycloalkyl;

m is 0, 1 or 2;

n is 0, 1, 2, 3 or 4;

p is 0, 1, 2, 3 or 4;

q is 0, 1 or 2;

r is 0 to 5;

s is 0 or 1;

t is 0 to 5;

u is 4 or 5; and

x is 0, 1, 2, 3or 4;

or the pharmaceutically acceptable salts or optical isomers thereof.

Another embodiment of the compounds of this invention are illustrated by the formula B:

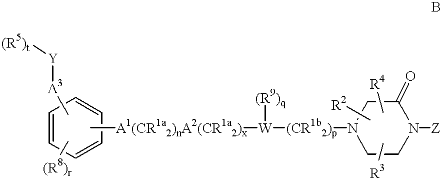

wherein:

R^{1a} and R^{1b} are independently selected from:

a) hydrogen,

b) unsubstituted or substituted aryl, unsubstituted or substituted heterocycle, unsubstituted or substituted C₃-C₁₀ cycloalkyl, R¹⁰O—, —N(R¹⁰)₂, or, C₂-C₈ alkenyl, or

c) unsubstituted or substituted C₁-C₆ alkyl wherein the sub stitutent on the substituted C₁-C₆ alkyl is selected from unsubstituted or substituted aryl, unsubstituted or substituted heterocycle, unsubstituted or substituted C₃-C₁₀ cycloalkyl, C₂-C₈ alkenyl, R¹⁰O—, or —N(R¹⁰)₂;

R² and R³ are independently selected from: H, unsubstituted or substituted C₁₋₆

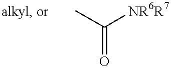

wherein the substituted group is substituted with one or more of:

1) aryl or heterocycle, unsubstituted or substituted with:

a) C₁-C₆ alkyl,

b) (CH₂)ₚOR⁶,

c) (CH₂)ₚNR⁶R⁷,

d) halogen,

e) CN;

2) C₃₋₆ cycloalkyl;

3) OR⁶;

4) SR^{6a}, S(O)R^{6a}, SO₂R^{6a},

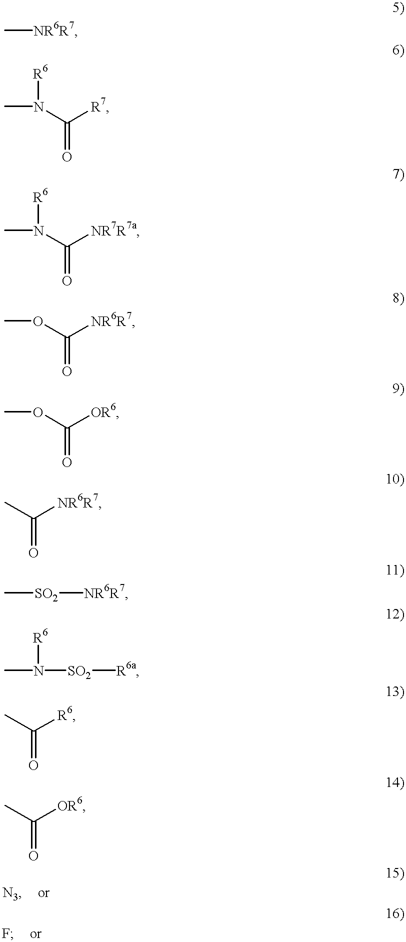

15) N₃, or

16) F; or

R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;

R⁴ is selected from H and unsubstituted or substituted C₁-C₆ alkyl;

and any two of R², R³ or R⁴ are optionally attached to the same carbon atom;

R⁵ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹ S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;

R⁶, R⁷ and R^{7a} are independently selected from: H, C₁-C₆ alkyl, C₃₋₆ cycloalkyl, heterocycle, aryl, aroyl, heteroaroyl, arylsulfonyl, heteroarylsulfonyl, unsubstituted or substituted with:

a) C₁₋₆ alkoxy,

b) C₁-C₂₀ alkyl

c) aryl or heterocycle,

d) halogen,

e) HO,

f) —C(O)R¹¹,

g) —SO₂R¹¹, or

h) N(R¹⁰)₂; or

R⁶ and R⁷ may be joined in a ring;

R⁷ and R^{7a} may be joined in a ring;

R⁶a is selected from: C₁-C₆ alkyl, C₃₋₆ cycloalkyl, heterocycle, aryl, aroyl, heteroaroyl, arylsulfonyl, heteroarylsulfonyl, unsubstituted or substituted with:

a) C₁₋₆ alkoxy,

b) C₁-C₂₀ alkyl

c) aryl or heterocycle,

d) halogen,

e) HO,

f) —C(O)R¹¹,

g) —SO₂R¹¹, or

h) N(R¹⁰)₂; or

R⁸ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;

R⁹ is selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ—,

R¹⁰C(O)NR¹⁰—, (R¹⁰)₂NC(O)—, (R¹⁰)₂NC(O)NR¹⁰—, R¹⁰2N—C(NR¹⁰)—, CN, NO₂, R¹⁰C(O)—, R¹⁰OC(O)—, N₃, —N(R¹⁰)₂, or R¹¹OC(O)NR¹⁰—, and

c) C₁-C₆ alkyl unsubstituted or substituted by aryl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;

R¹⁰ is independently selected from hydrogen, unsubstituted or substituted C₁-C₆ alkyl, perfluoroalkyl, unsubstituted or substituted aralkyl, and unsubstituted or substituted aryl;

R¹¹ is independently selected from unsubstituted or substituted C₁-C₆ alkyl and unsubstituted or substituted aryl;

A¹ and A² are independently selected from: a bond, —CH═CH—, —C≡C—, —C(O)—, —C(O)NR¹⁰—, —NR¹⁰C(O)—, O, —N(R¹⁰)—, —S(O)₂N(R¹⁰)—, —N(R¹⁰)S(O)₂—, or S(O)ₘ;

A³ is selected from —C(O)—, —C(R^{1a})₂—, O, —N(R¹⁰)— and S(O)ₘ;

W is a heterocycle selected from imidazolyl, pyridyl, thiazolyl, indolyl, quinolinyl, isoquinolinyl and thienyl;

Y is heteroaryl;

Z is a unsubstituted or substituted group selected from aryl, heteroaryl, arylmethyl, heteroarylmethyl, arylsulfonyl, heteroarylsulfonyl, wherein the substituted group is substituted with one or more of the following:

1) C₁-C₆ alkyl, unsubstituted or substituted with:

a) C₁₋₆ alkoxy,

b) NR⁶R⁷,

c) C₃₋₆ cycloalkyl,

d) aryl or heterocycle,

e) HO,

f) —S(O)ₘR^{6a}, or

g) —C(O)NR⁶R⁷,

2) unsubstituted or substituted aryl or unsubstituted or substituted heterocycle,

3) halogen,

4) OR⁶,

5) NR⁶R⁷,

6) CN,

7) NO₂,

8) CF₃;

9) —S(O)ₘR^{6a},

10) —C(O)NR⁶R⁷,

11) C₃-C₆ cycloalkyl,

12) —OCF₃, or

13) unsubstituted or substituted C₁₋₆ alkoxy;

m is 0, 1 or 2;

n is 0, 1, 2, 3 or 4;

p is 0, 1, 2, 3or 4;

q is 0, 1 or 2;

r is 0 to 5;

t is 0 to 5;

u is 4 or 5; and

x is 0, 1, 2, 3or 4;

or the pharmaceutically acceptable salts or optical isomers thereof.

Another embodiment of the compounds of this invention are illustrated by the formula C:

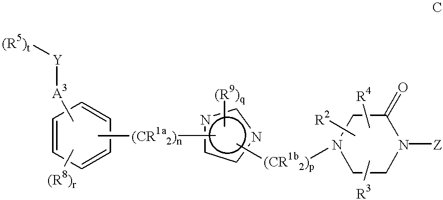

wherein: R^{1a} and R^{1b} are independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, R¹⁰O—, or —N(R¹⁰)₂, or

c) unsubstituted or substituted C₁-C₆ alkyl wherein the substitutent on the substituted C₁-C₆ alkyl is selected from unsubstituted or substituted aryl, unsubstituted or substituted heterocycle, unsubstituted or substituted C₃-C₁₀ cycloalkyl, C₂-C₈ alkenyl, R¹⁰O—, or —N(R¹⁰)₂;

R² is H, unsubstituted or substituted C₁₋₆ alkyl, or

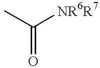

wherein the substituted group is substituted with one or more of:

1) aryl,

2) heterocycle,

3) OR⁶,

4) SR^{6a}, SO₂R^{6a}, or

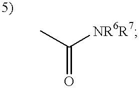

R³ and R⁴ are independently selected from H and unsubstituted or substituted C₁-C₆ alkyl;

and any two of R², R³ or R⁴ are optionally attached to the same carbon atom;

R⁵ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹1OC(O)NR¹⁰—;

R⁶ and R⁷ are independently selected from: H, C₁-C₆ alkyl, C₃₋₆ cycloalkyl, heterocycle, aryl, unsubstituted or substituted with:

a) C₁₋₆ alkoxy,

b) C₁-C₂₀ alkyl

c) aryl or heterocycle,

d) halogen, or

e) HO;

R⁶ and R⁷ may be joined in a ring;

R^{6a} is selected from: C₁-C₆ alkyl, C₃₋₆ cycloalkyl, heterocycle, aryl, aroyl, heteroaroyl, arylsulfonyl, heteroarylsulfonyl, unsubstituted or substituted with: a) C₁₋₆ alkoxy,

b) C₁-C₂₀ alkyl

c) aryl or heterocycle,

d) halogen, or

e) HO;

R⁸ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹⁰OC(O)NR¹⁰—;

R⁹ is selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;

R¹⁰ is independently selected from hydrogen, unsubstituted or substituted C₁-C₆ alkyl, perfluoroalkyl, unsubstituted or substituted aralkyl, and unsubstituted or substituted aryl;

R¹¹ is independently selected from unsubstituted or substituted C₁-C₆ alkyl and unsubstituted or substituted aryl;

A³ is selected from —C(O)—, —C(R^{1a})₂—, O, —N(R¹⁰)— and S(O)ₘ;

Y is heteroaryl;

Z is a unsubstituted or substituted group selected from aryl, heteroaryl, arylmethyl, heteroarylmethyl, wherein the substituted group is substituted with one or more of the following:

1) C₁-C₆ alkyl, unsubstituted or substituted with:

a) C₁₋₆ alkoxy,

b) NR⁶R⁷,

c) C₃₋₆ cycloalkyl,

d) aryl or heterocycle,

e) HO,

f) —S(O)ₘR^{6a}, or

g) —C(O)NR⁶R⁷,

2) unsubstituted or substituted aryl or unsubstituted or substituted heterocycle,

3) halogen,

4) OR⁶,

5) NR⁶R⁷,

6) CN,

7) NO₂,

8) CF₃;

9) —S(O)ₘR^{6a},

10) —C(O)NR⁶R⁷,

11) C₃-C₆ cycloalkyl,

12) —OCF₃, or

13) unsubstituted or substituted C₁₋₆ alkoxy;

m is 0, 1 or 2;

n is 0, 1, 2, 3or 4;

p is 0, 1, 2, 3or 4;

q is 0, 1 or 2;

r is 0 to 5;

t is 0 to 5; and

u is 4 or 5;

or the pharmaceutically acceptable salts or optical isomers thereof.

Specific examples of the compounds of this invention are as follows:

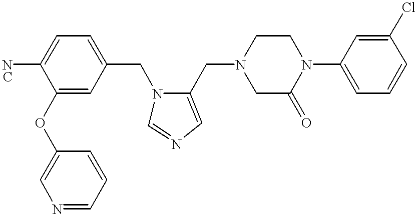

1-(3-chlorophenyl)-4-[1-(3-(3-pyridyloxy)-4-cyanobenzyl)-5-imidazolylmethyl]-2-piperazinone;

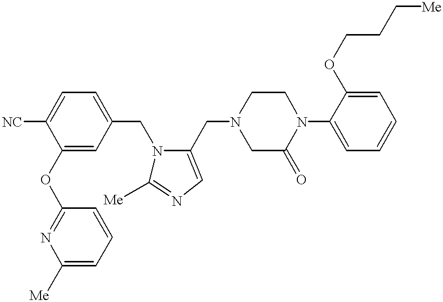

1-(2-(n-Butyloxy)phenyl)-4-[1-(3-((6-methyl-2-pyridyl)oxy)-4-cyanobenzyl)-2-methyl-5-imidazolylmethyl]-2-piperazinone;

or the pharmaceutically acceptable salts or optical isomers thereof.

The compounds of the instant invention differ from previously disclosed piperazinone-containing and piperazine-containing compounds, (PCT Publication No. WO 96/30343—Oct. 3, 1996; PCT Publication No. WO 96/31501, Oct. 10, 1996; PCT Publication No. WO 97/36593, Oct. 9, 1997; PCT Publication No. WO 97/36592, Oct. 9, 1997) that were described as inhibitors of farnesyl-protein transferase (FPTase), in that, among other things, the instant compounds are dual inhibitors of famesyl-protein transferase and geranylgeranyl-protein transferase type I (GGTase-I).

The compounds of the instant invention are further characterized in that the inhibitory activity of the compounds against FPTase is greater than the inhibitory activity against GGTase-I. Preferably, the compounds of the instant invention inhibit FPTase in vitro (Example 3) at an IC₅₀ of less than 100 nM and inhibit GGTase-I in vitro (Example 4) at an IC₅₀ of less than 5 μM. Preferably, the compounds of the instant invention inhibit the cellular processing of the hDJ protein (Example 8) at an EC₅₀ of less than about 250 nM. Also preferably, the compounds of the instant invention inhibit the cellular processing of the Rap1 protein (Example 9) at an EC₅₀ of less than about 10 μM. More preferably, the compounds of the instant invention inhibit the cellular processing of the Rap1 protein (Example 9) at an EC₅₀ of less than about 1 μM. Also more preferably, the ratio of the IC₅₀ of the compounds of this embodiment of the instant invention for in vitro inhibition of GGTase type I to the IC₅₀ of the compounds of the instant invention for in vitro inhibition of FPTase is greater than 1 and less than 25. Also more preferably, the ratio of the EC₅₀ of the compounds of the instant invention for inhibition of the cellular processing of the hDJ protein (Example 8) to the EC₅₀ of the compounds of the instant invention for inhibition of the cellular processing of the Rap1 protein is between about 1 and about 100.

The compounds of the present invention may have asymmetric centers and occur as racemates, racemic mixtures, and as individual diastereomers, with all possible isomers, including optical isomers, being included in the present invention. When any variable (e.g. aryl, heterocycle, R^{1a}, R² etc.) occurs more than one time in any constituent, its definition on each occurrence is independent at every other occurrence. Also, combinations of substituents/or variables are permissible only if such combinations result in stable compounds.

As used herein, “alkyl” is intended to include both branched and straight-chain saturated aliphatic hydrocarbon groups having from 1 to 10 carbon atoms, unless otherwise specified; “alkoxy” represents an alkyl group having from 1 to 6 carbon atoms, unless otherwise specified, attached through an oxygen bridge. “Halogen” or “halo” as used herein means fluoro, chloro, bromo and iodo.

As used herein, “cycloalkyl” is intended to include non-aromatic hydrocarbon groups having from 3 to 10 carbon atoms, unless otherwise specified. Examples of such cycloalkyl groups includes, but are not limited to, cyclopropyl, cyclobutyl, cyclohexyl, cycloheptyl, cyclooctyl, admantyl and the like.

If no number of carbon atoms is specified, the term “alkenyl” refers to a non-aromatic hydrocarbon, straight, branched or cyclic, containing from 2 to 10 carbon atoms, unless otherwise indicated, and at least one carbon to carbon double bond. Preferably one carbon to carbon double bond is present, and up to four non-aromatic carbon-carbon double bonds may be present. Thus, “C₂-C₈ alkenyl” means an alkenyl radical having from 2 to 8 carbon atoms. Examples of such alkenyl groups include, but are not limited to, ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms, unless otherwise indicated, and at least one carbon to carbon triple bond. Up to three carbon-carbon triple bonds may be present. Thus, “C₂-C₈ alkynyl” means an alkynyl radical having from 2 to 8 carbon atoms. Examples of such alkynyl groups include, but are not limited to, ethynyl, propynyl and butynyl. As described above with respect to alkyl, the straight, branched or cyclic portion of the alkynyl group may contain triple bonds and may be substituted if a substituted alkynyl group is indicated.

As used herein, “aryl” is intended to mean any stable monocyclic or bicyclic carbon ring of up to 7 members in each ring, wherein at least one ring is aromatic. Examples of such aryl elements include, but are not limited to, phenyl, naphthyl, tetrahydronaphthyl, indanyl, biphenyl, phenanthryl, anthryl, acenaphthyl and the like.

As used herein, “aralkyl” is intended to mean an aryl moiety, as defined above, attached through a C₁-C₆ alkyl linker, where alkyl is defined above. Examples of aralkyls include, but are not limited to, benzyl, naphthylmethyl and phenylbutyl.

The term heterocycle or heterocyclic, as used herein, represents a stable 5- to 7-membered monocyclic or stable 8- to 1 1-membered bicyclic hetero-cyclic ring which is either saturated or unsaturated, and which consists of carbon atoms and from one to four heteroatoms selected from the group consisting of N, O, and S, and including any bicyclic group in which any of the above-defined heterocyclic rings are fused to a benzene ring. The term heterocycle or heterocyclic includes heteroaryl moieties. The heterocyclic ring may be attached at any heteroatom or carbon atom which results in the creation of a stable structure. Examples of such heterocyclic elements include, but are not limited to, azepinyl, benzimidazolyl, benzisoxazolyl, benzofurazanyl, benzopyranyl, benzopyrazolyl, benzotriazolyl, benzothiopyranyl, benzofuryl, benzothiazolyl, benzothienyl, benzoxazolyl, chromanyl, cinnolinyl, dihydrobenzofuryl, dihydrobenzofuranyl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, morpholinyl, naphthyridinyl, oxadiazolyl, 2-oxoazepinyl, 4-oxonaphthyridinyl, oxazolyl, 2-oxopiperazinyl, 2-oxopiperdinyl, 2-oxopyrrolidinyl, 2-oxopyridyl, 2-oxoquionolinyl, piperidyl, piperazinyl, pyridyl, pyridinyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydrofuranyl, tetrahydroimidazopyridinyl, tetrahydroisoquinolinyl, tetrahydroquinolinyl, thiamorpholinyl, thiamorpholinyl sulfoxide, thiazolyl, thiazolinyl, thienofuryl, thienothienyl, thienyl, triazolyl, and the like.

As used herein, “heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O, and S. Examples of such heteroaryl elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyridyl N-oxide, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and the like.

As used herein, “heteroaralkyl” is intended to mean a heteroaryl moiety, as defined above, attached through a C₁-C₆ alkyl linker, where alkyl is defined above. Examples of heteroaralkyls include, but are not limited to, 2-pyridylmethyl, 2-morpholinylethyl, 2-imidazolylethyl, 2-quinolinylmethyl, 2-imidazolylmethyl, 1-piperazineethyl, and the like.

As used herein, the terms “substituted alkyl”, “substituted alkenyl”, “substituted alkynyl” and “substituted alkoxy” are intended to include the branch or straight-chain alkyl group of the specified number of carbon atoms, wherein the carbon atoms may be substituted with F, Cl, Br, I, CF₃, OCF₃, CN, N₃, NO₂, NH₂, N(C₁-C₆ alkyl)₂, oxo, OH, —O(C₁-C₆ alkyl), S(O)₀₋₂, (C₁-C₆ alkyl)S(O)₀₋₂—, C₂-C₆ alkenyl, C₂-C₆ alkynyl, —(C₁-C₆ alkyl)S(O)₀₋₂(C₁-C₆ alkyl), C₃-C₂₀ cycloalkyl, —C(O)NH₂, HC(O)NH—(C₁-C₆ alkyl)C(O)NH—, H₂NC(O)NH—(C₁-C₆ alkyl)C(O)—, —O(C₁-C₆ alkyl)CF₃, (C₁-C₆ alkyl)OC(O)—, (C₁-C₆ alkyl)O(C₁-C₆ alkyl)—, (C₁-C₆ alkyl)C(O)₂(C₁-C₆ alkyl)—, (C₁-C₆ alkyl)OC(O)NH—, aryl, heterocycle, aralkyl, heteroaralkyl, halo-aryl, halo-aralkyl, halo-heterocycle, halo-heteroaralkyl, cyano-aryl, cyano-aralkyl, cyano-heterocycle and cyano-heteroaralkyl.

As used herein, the terms “substituted aryl”, “substituted heterocycle”, “substituted heteroaryl”, “substituted cycloalkyl”, “substituted benzyl”, “substituted aralkyl” and “substituted heteroaralkyl” are intended to include the cyclic group containing from 1 to 3 substitutents in addition to the point of attachment to the rest of the compound. Such substitutents are preferably selected from the group which includes but is not limited to F, Cl, Br, I, CF₃, OCF₃, NH₂, N(C₁-C₆ alkyl)₂, NO₂, CN, N₃, C₁-C₂₀ alkyl, C₃-C₂₀ cycloalkyl, —OH, —O(C₁-C₆ alkyl), S(O)₀₋₂, (C₁-C₆ alkyl)S(O)₀₋₂—, (C₁-C₆ alkyl)S(O)₀₋₂(C₁-C₆ alkyl)—, —C(O)NH₂, HC(O)NH—, (C₁-C₆ alkyl)C(O)NH—, H₂NC(O)NH—, (C₁-C₆ alkyl)C(O)—, (C₁-C₆ alkyl)OC(O)—, (C₁-C₆ alkyl)O(C₁-C₆ alkyl)—, (C₁-C₆)C(O)₂(C₁-C₆ alkyl)—, (C₁-C₆ alkyl)OC(O)NH—, aryl, aralkyl, heterocycle, heteroaralkyl, halo-aryl, halo-aralkyl, haloheterocycle, haloheteroaralkyl, cyano-aryl, cyano-aralkyl, cyano-heterocycle and cyanohetero-aralkyl.

As used herein in the definition of R² and R³, the term “the substituted group” is intended to mean a substituted C₁₋₆ alkyl, substituted C₂₋₈ alkenyl, substituted C₂₋₈ alkynyl, substituted aryl or substituted heterocycle.

As used herein in the definition of R⁶, R^{6a}, R⁷ and R^{7a}, the substituted C₁₋₈ alkyl, substituted C₃₋₆ cycloalkyl, substituted aroyl, substituted aryl, substituted heteroaroyl, substituted arylsulfonyl, substituted heteroarylsulfonyl and substituted heterocycle include moieties containing from 1 to 3 substituents in addition to the point of attachment to the rest of the compound. Preferably, such substituents are selected from the group which includes but is not limited to F, Cl, Br, CF₃, NH₂, N(C₁-C₆ alkyl)₂, NO₂, CN, (C₁-C₆ alkyl)O—, —OH, (C₁-C₆ alkyl)S(O)ₘ—, (C₁-C₆ alkyl)C(O)NH—, (C₁-C₆ alkyl)C(O)—, (C₁-C₆ alkyl)OC(O)—, N₃, (C₁-C₆ alkyl)OC(O)NH—, phenyl, pyridyl, imidazolyl, oxazolyl, isoxazolyl, thiazolyl, thienyl, furyl, isothiazolyl and C₁-C₂₀ alkyl.

As used herein, examples of “C₃-C₂₀ cycloalkyl” may include, but are not limited to:

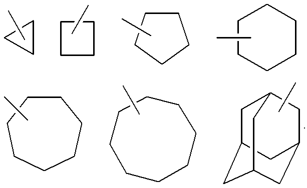

When R² and R³ are combined to form —(CH₂)ᵤ—, cyclic moieties are formed. Examples of such cyclic moieties include, but are not limited to:

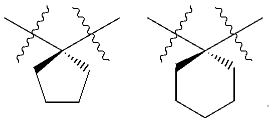

In addition, such cyclic moieties may optionally include a heteroatom(s). Examples of such heteroatom-containing cyclic moieties include, but are not limited to:

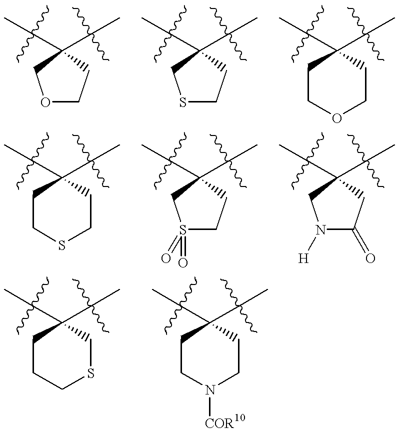

The moiety formed when, in the definition of R⁶ and R⁷ or R⁶ and R^{7a} are joined to form a ring, is illustrated by, but not limited to, the following:

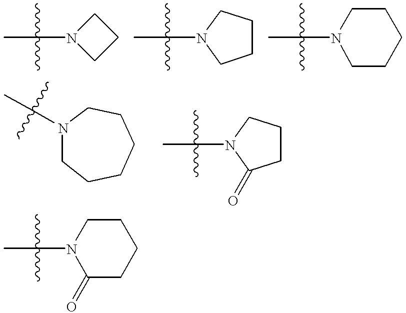

Lines drawn into the ring systems from substituents (such as from R², R³, R⁴ etc.) indicate that the indicated bond may be attached to any of the substitutable ring carbon atoms or heteroatoms.

Preferably, R^{1a} and R^{1b} are independently selected from: hydrogen, aryl, heterocycle, CN, —N(R¹⁰)₂, (R¹⁰)₂NC(O)—, R¹⁰C(O)NR¹⁰— or unsubstituted or substituted C₁-C₆ alkyl. More preferably, R^{1a} and R^{1b} are independently selected from: hydrogen, —N(R¹⁰)₂ or unsubstituted or substituted C₁-C₆ alkyl.

Preferably, R² is selected from: hydrogen, unsubstituted or substituted C₁₋₆ alkyl,

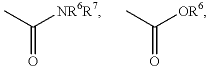

unsubstituted or substituted C₂₋₈ alkenyl and unsubstituted or substituted C₂₋₈ alkynyl.

Preferably R³ and R⁴ are independently selected from H and unsubstituted or substituted C₁-C₆ alkyl. Most preferably, R³ and R⁴ are H.

Preferably, R⁵ is selected from H, halo, unsubstituted or substituted C₁₋₆ alkyl, unsubstituted or substituted C₁₋₆ alkoxy, unsubstituted or substituted aryl, CN, NO₂, R¹⁰C(O)NR¹⁰—, —OR and (R¹⁰)₂NC(O)—. More preferably, is selected from H, halo, unsubstituted or substituted C₁₋₆ alkyl, unsubstituted or substituted C₁₋₆ alkoxy, and unsubstituted or substituted aryl.

Preferably, R⁶, R⁷ and R^{7a} are independently selected from: hydrogen, unsubstituted or substituted C₁-C₆ alkyl, unsubstituted or substituted aryl and unsubstituted or substituted cycloalkyl.

Preferably, R^{6a} is selected from unsubstituted or substituted C₁-C₆ alkyl, unsubstituted or substituted aryl and unsubstituted or substituted cycloalkyl.

Preferably, R⁸ is selected from H, halo, unsubstituted or substituted C₁₋₆ alkyl, unsubstituted or substituted C₁₋₆ alkoxy, unsubstituted or substituted aryl, CN, NO₂, R¹⁰C(O)NR¹⁰—, —OR ¹⁰and (R¹⁰)₂NC(O)—. Most preferably, r is 1 to 3 and at least one R⁸ is CN.

Preferably, R⁹ is selected from hydrogen, halo or unsubstituted or substituted C₁-C₆ alkyl.

Preferably, R¹⁰ is selected from H, C₁-C₆ alkyl, benzyl and aryl.

Preferably, A¹ and A² are independently selected from: a bond, —C(O)NR¹⁰—, —NR¹⁰C(O)—, , —N(R¹⁰)—, —S(O)₂N(R¹⁰)— and —N(R¹⁰)S(O)₂—. Most preferably, A¹ and A² are a bond.

Preferably, A³ is selected from: —O—, -(CR^{1a})₂—, and —C(O)—.

Preferably, V is aryl. Most preferably, V is phenyl or naphthyl.

Preferably, W is selected from imidazolyl, oxazolyl, pyrazolyl, pyyrolidinyl, pyridinyl, thiazolyl, indolyl, quinolinyl, and isoquinolinyl. More preferably, W is selected from imidazolyl and pyridinyl.

Preferably, Y is pyridyl, imidazolyl, pyrimidinyl, pyrazinyl or indolyl. More preferably, Y is pyridyl or imidazolyl.

Preferably, Z is selected from unsubstituted or substituted aryl, unsubstituted or substituted heteroaryl, and unsubstituted or substituted arylmethyl. Most preferably, Z is selected from unsubstituted or substituted phenyl, unsubstituted or substituted pyridyl or 1,2 methylenedioxybenzene.

Preferably, n and x are independently 0, 1, or 2.

Preferably p is 1, 2 or 3.

Preferably, q is 0 or 1.

Preferably, r and t are independently selected from 0, 1, 2 or 3.

Preferably s is 0.

Preferably, the moiety

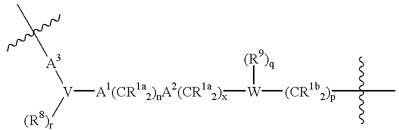

is selected from:

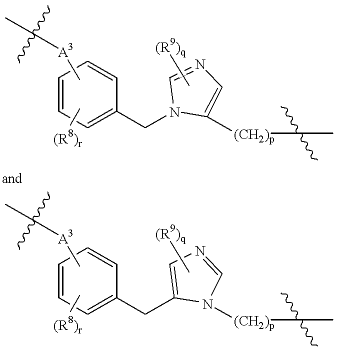

Preferably, the moiety —A¹ (CR^{1a}₂)ₙA²(CR^{1a}₂)ₓ— is not a bond.

It is intended that the definition of any substituent or variable (e.g., R^{1a}, R⁸, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials.

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds of this invention can be synthesized from the compounds of this invention which contain a basic moiety by conventional chemical methods. Generally, the salts are prepared either by ion exchange chromatography or by reacting the free base with stoichiometric amounts or with an excess of the desired salt-forming inorganic or organic acid in a suitable solvent or various combinations of solvents.

Abbreviations which may be used in the description of the chemistry and in the Examples that follow include:

| | |
|---|---|
| Ac₂O | Acetic anhydride; |
| AIBN | 2,2′-Azobisisobutyronitrile; |
| BOC/Boc | t-Butoxycarbonyl; |
| CBz | Carbobenzyloxy; |
| DBAD | Di-tert-butyl azodicarboxylate; |
| DBU | 1,8-Diazabicyclo[5.4.0]undec-7-ene; |
| DCE | 1,2-Dichloroethane; |
| DIEA | N,N-Diisopropylethylamine; |
| DMAP | 4-Dimethylaminopyridine; |
| DME | 1,2-Dimethoxyethane; |
| DMF | N,N-Dimethylformamide; |
| DMSO | Methyl sulfoxide; |
| DPPA | Diphenylphosphoryl azide; |
| DTT | Dithiothreitol; |
| EDC | 1-(3-Dimethylaminopropyl)-3-ethyl-carbodiimide- |
| | hydrochloride; |
| EDTA | Ethylenediaminetetraacetic acid; |
| Et₃N | Triethylamine; |
| EtOAc | Ethyl acetate; |
| EtOH | Ethanol; |
| FAB | Fast atom bombardment; |
| HEPES | 4-(2-Hydroxyethyl)-1-piperazineethanesulfonic acid; |
| HOBT | 1-Hydroxybenzotriazole hydrate; |
| HOOBT | 3-Hydroxy-1,2,2-benzotriazin-4(3H)-one; |
| HPLC | High-performance liquid chromatography; |
| LAH | Lithium aluminum hydride; |
| MCPBA | m-Chloroperoxybenzoic acid; |
| Me | Methyl; |
| MeOH | Methanol; |
| Ms | Methanesulfonyl; |
| MsCl | Methanesulfonyl chloride; |
| n-Bu₃P | Tri-n-butylphosphine; |
| NaHMDS | Sodium bis(trimethylsilyl)amide; |
| NBS | N-Bromosuccinimide; |
| Ph | phenyl; |
| PMSF | a-Toluenesulfonyl chloride; |
| Py or pyr | Pyridine; |
| PYBOP | Benzotriazole-1-yl-oxy-trispyrrolidinophosphonium |
| | hexafluorophosphate; |
| t-Bu | tert-Butyl; |
| TBAF | Tetrabutylammoniumfluoride; |
| RPLC | Reverse Phase Liquid Chromatography; |
| TBSCl | tert-Butyldimethylsilyl chloride; |
| TFA | Trifluoroacetic acid; |
| THF | Tetrahydrofuran; |
| TMS | Tetramethylsilane; and |
| Tr | Trityl; |

The reactions described herein may be employed in a linear sequence to provide the compounds of the invention or they may be used to synthesize fragments which are subsequently joined by the alkylation reactions described in the Schemes. The procedures discussed and illustrated in the following schemes and synopsis may be used in the preparation of the compounds of the instant invention, for either (R) or (S) stereochemistry.

Reactions used to generate the compounds of this invention are prepared by employing reactions as shown in the Schemes 1-15, in addition to other standard manipulations such as ester hydrolysis, cleavage of protecting groups, etc., as may be known in the literature or exemplified in the experimental procedures. In the Schemes below, R represents aryl or heteroaryl, X represents a halide, Rsub represents a substitution on the Z substituent and Ar represents an aryl. However, the point of attachment of any of the substituents to the ring is illustrative only and is not meant to be limiting.

These reactions may be employed in a linear sequence to provide the compounds of the invention or they may be used to synthesize fragments which are subsequently joined by the alkylation reactions described in the Schemes.

Synopsis of Schemes 1-15:

The requisite intermediates are in some cases commercially available, or can be prepared according to literature procedures, for the most part. In Scheme 1, for example, the synthesis of unsubstituted piperazinones is outlined. Arylamine 1 in methylene chloride at 0° C. is added to an acidic solution of 1,4-dioxane. The resulting product is combined with 2-oxazolidinone to give diamine 2. Diamine 2 is protected with di-tert-butylpyrocarbonate to give the Boc protected diamine 3 which is reacted with chloroacetyl chloride in CH₂Cl₂ at 0° C. to yield chloroacetamide 4. Chloroacetamide IV is cyclized to the corresponding Boc protected piperazinone 5 by heating in DMF and K₂CO₃. The Boc protected piperazinone is then deprotected with acid, for example hydrogen chloride in chloroform or ethyl acetate, or trifluoroacetic acid in methylene chloride to give unsubstituted piperazinone 6.

Preparation of 5-substituted piperazin-2-ones is illustrated in Scheme 2 in which aldehyde 7 is reductively alkylated with an aryl amine and the resulting product is converted to the Boc protected substituted piperazinone 8 by acylation with chloroacetylchloride followed by base-induced cyclization. Deprotection under standard conditions gives substituted piperazinone 9.

Scheme 3 depicts the preparation of fluorobenzonitrilealdehyde 15. 4-bromo-3-fluorotoluene 10 in DMF is reacted with Zn(CN)₂ and PD(PPh₃)₄. The resulting product is treated with N-bromosuccinamide and benzoylperoxide to give 4-cyano-3-fluoro benzyl bromide 11. Acetoxymethyl-imidazole 13 is prepared by combining 11 with protected imidazole acetate 12 in EtOAc at reflux. The acetate 13 is hydrolized to the corresponding alcohol with LiOH/water and oxidized to aldehyde 15 under standard oxidation conditions. Aldehyde 15 can be reductively alkylated with a variety of amines such as unsubstitited piperazinone 6 (Scheme 4) or substituted piperazinone 9. The resulting intermediates such as 16 can be converted into final products 17 via base-promoted addition reactions as depicted in Scheme 4.

As shown in Scheme 5, the piperazinone intermediate 9 can be reductively alkylated with other aldehydes such as 1-trityl-4-imidazolylcarboxaldehyde or 1-trityl-4-imidazolylacetaldehyde, to give products such as 18. The trityl protecting group can be removed from 18 to give 19, or alternatively, 18 can first be treated with an alkyl halide then subsequently deprotected to give the alkylated imidazole 20. Alternatively, the intermediate 9 can be acylated or sulfonylated by standard techniques.

The isomeric substituted piperazin-3-ones can be prepared as described in Scheme 6. The imine formed from arylcarboxamides 21 and 2-aminoglycinal diethyl acetal 22 can be reduced under a variety of conditions, including sodium triacetoxyborohydride in dichloroethane, to give the amine 23. Amino acids can be coupled to amines 23 under standard conditions, and the resulting amide 24 when treated with aqueous acid in tetrahydrofuran can cyclize to the unsaturated 25. Catalytic hydrogenation under standard conditions gives the requisite intermediate 26, which may be used to prepare compounds of the instant invention, utilizing techniques described herein.

Scheme 7 illustrates the use of an optionally substituted homoserine lactone 27 to prepare a Boc-protected piperazinone 28. Intermediate 28 may be deprotected and reductively alkylated or acylated as illustrated in the previous Schemes. Alternatively, the hydroxyl moiety of intermediate 28 may be mesylated and displaced by a suitable nucleophile, such as the sodium salt of ethane thiol, to provide an intermediate 29. Intermediate 28 may also be oxidized to provide the carboxylic acid on intermediate 30, which can be utilized form an ester or amide moiety.

Amino acids of the general formula 32 which have a sidechain not found in natural amino acids may be prepared by the reactions illustrated in Scheme 8 starting with the readily prepared imine 31.

Schemes 9-12 illustrate syntheses of suitably substituted aldehydes useful in the syntheses of the instant compounds wherein the variable W is present as a pyridyl moiety. Similar synthetic strategies for preparing alkanols that incorporate other heterocyclic moieties for variable W are also well known in the art.

Scheme 13 depicts the synthesis of compounds of the instant invention having an ethyl linker between the imidazolyl moiety and the piperazinone moiety. Activated zinc is added to a fluoroaryl methylhalide in THF to form the arylmethyl zinc halide, which is subsequently coupled to an N-protected 4-iodoimidazole to give compound 33. Regiospecfic alkylation of the imidazole ring is accomplished with ethyl bromoacetate, with subsequent methanolysis of the intermediate imidazolium salt giving 34. Elaboration of 34 to the primary amine 38 proceeds through standard chemistry. Alkylation of the amine with suitably substituted N-aryl chloroaceamide provides the intermediate amide 39, which can be reductively alkyated with glycol aldehyde dimer to give hydroxyethyl compound 40. Ring closure under Mitsunobu conditions furnishes piperazinone 41.

Scheme 14 illustrates the synthetic strategy that is employed when the R⁸ substitutent is not an electronic withdrawing moiety either ortho or para to the fluorine atom. In the absence of the electronic withdrawing moiety, the alkylation can be accomplished via an Ullmann reaction. Thus, the imidazolylmethylacetate 12 is treated with a suitably substituted halobenzylbromide to provide the 1-benzylimidazolyl intermediate 42. The acetate functionality of intermediate 42 was converted to an aldehyde which was then reductively coupled to intermediate 6, prepared as illustrated in Scheme 1. Coupling under standard Ullmann conditions provided compound 45 of the instant invention.

Scheme 15 illustrates the preparation of a substituted aryl or heteoraryl on the right side of the piperazinone. 4-Benzyloxycaronyl-2-piperazinone 46 is commercially available and can be N-alkylated after deprotonation with NaH to provide compound 48, or can be N-arylated in a copper-promoted coupling reaction to provide compound 50.

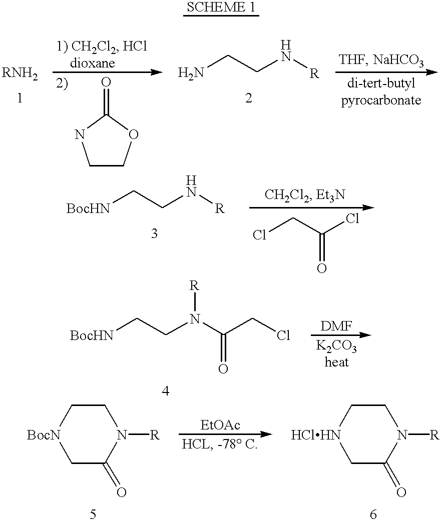

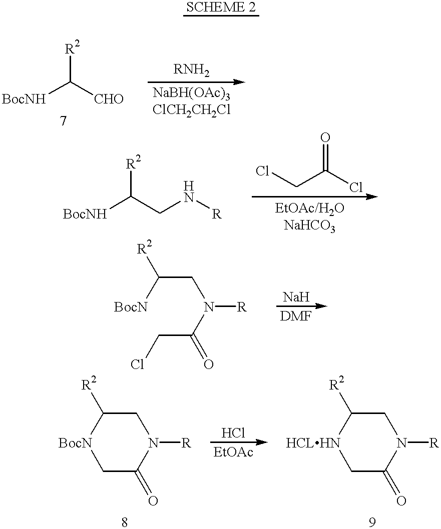

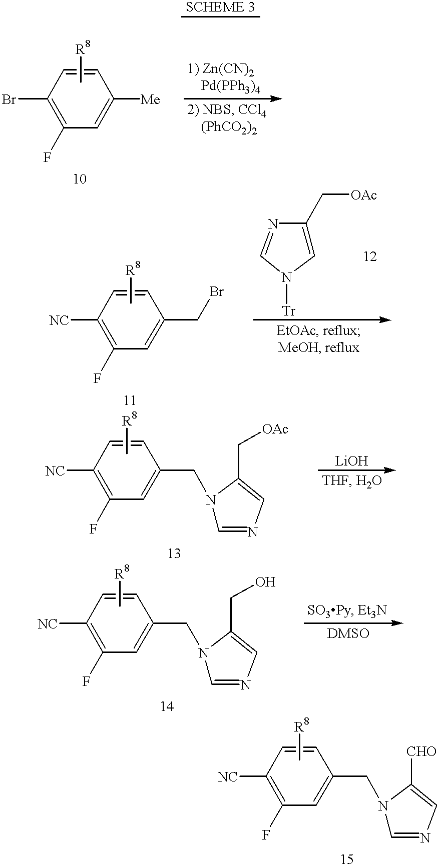

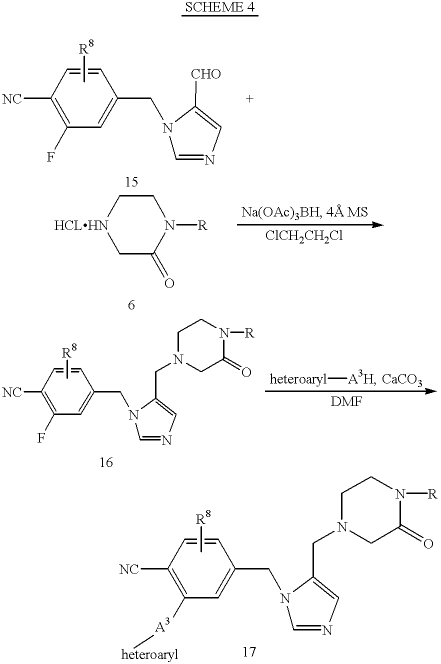

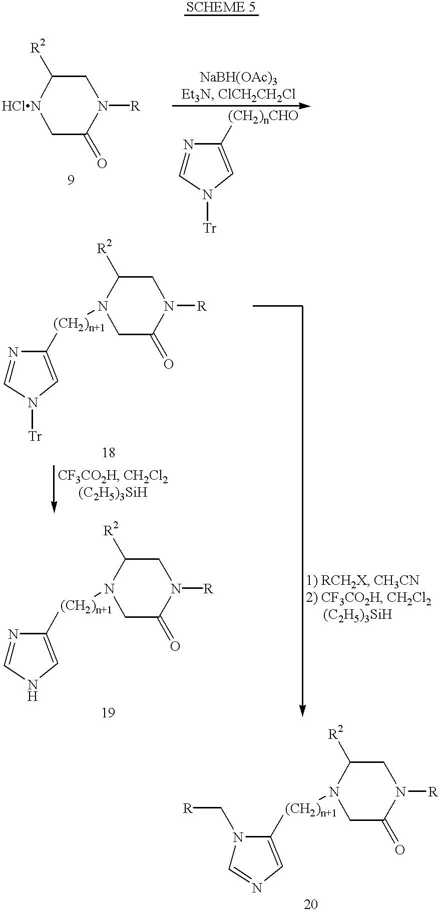

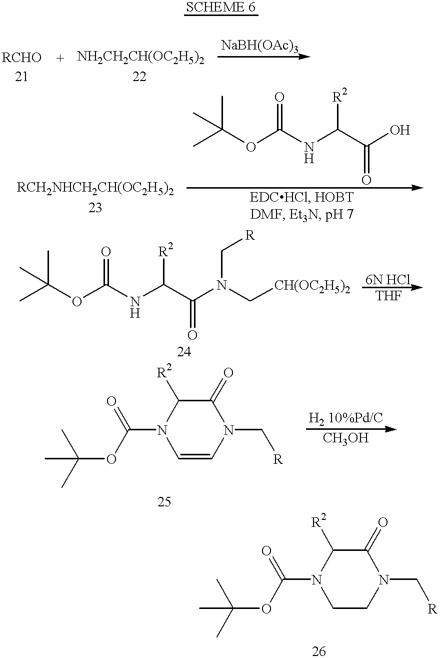

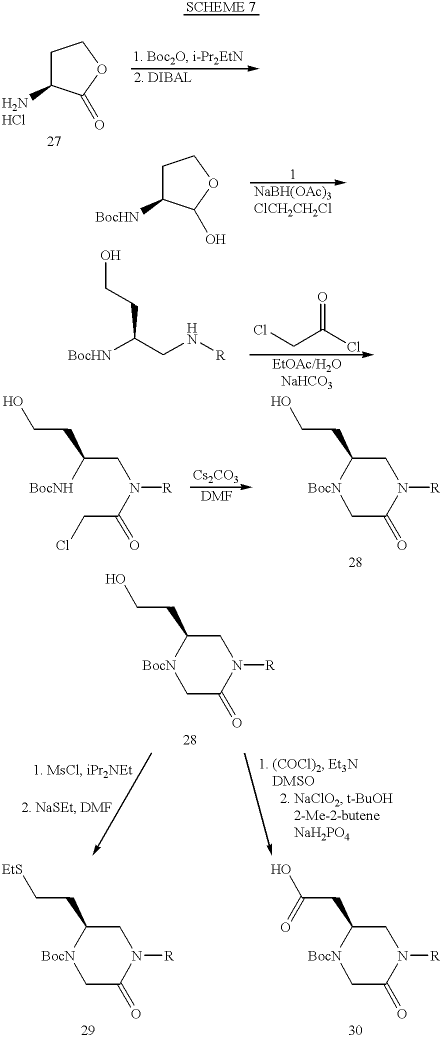

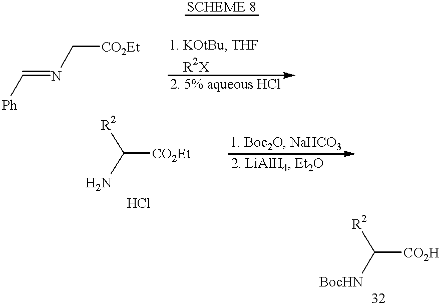

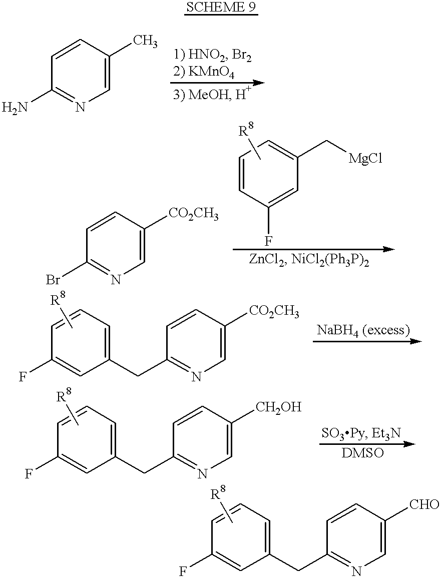

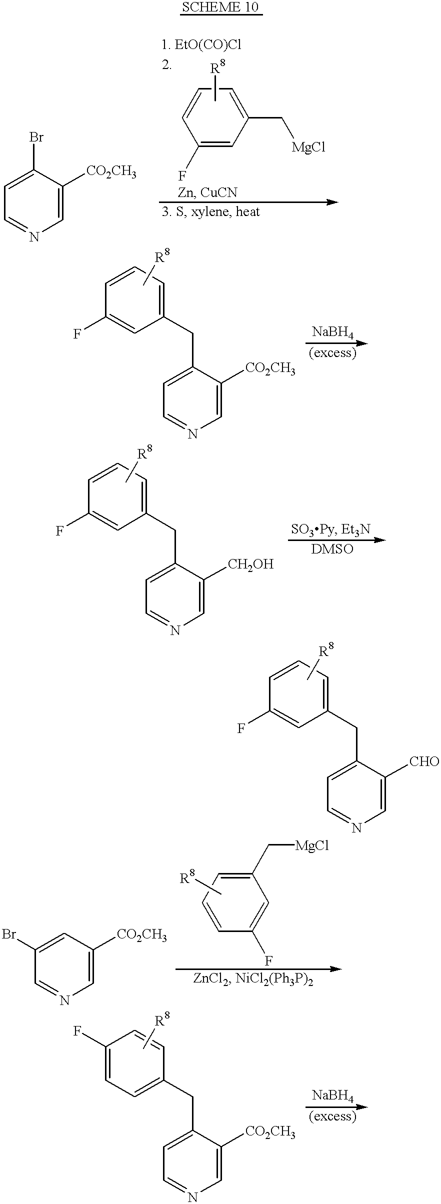

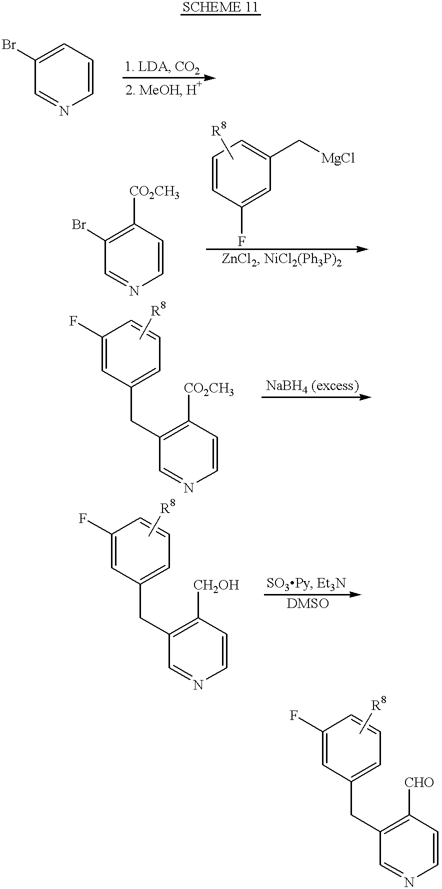

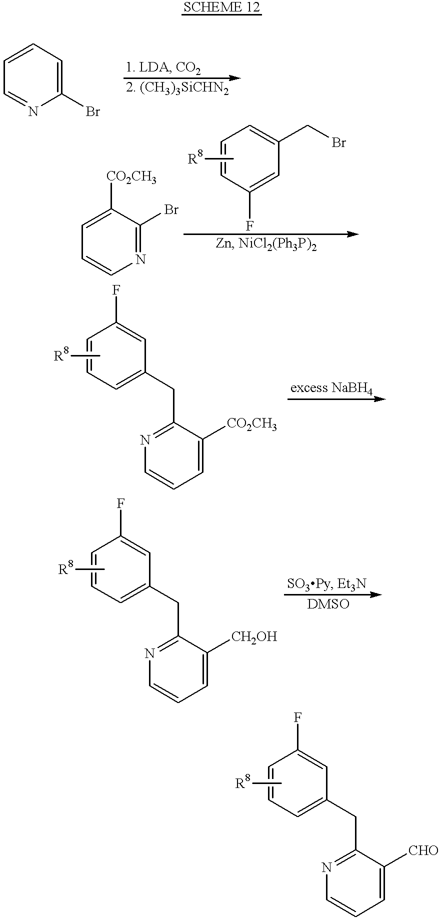

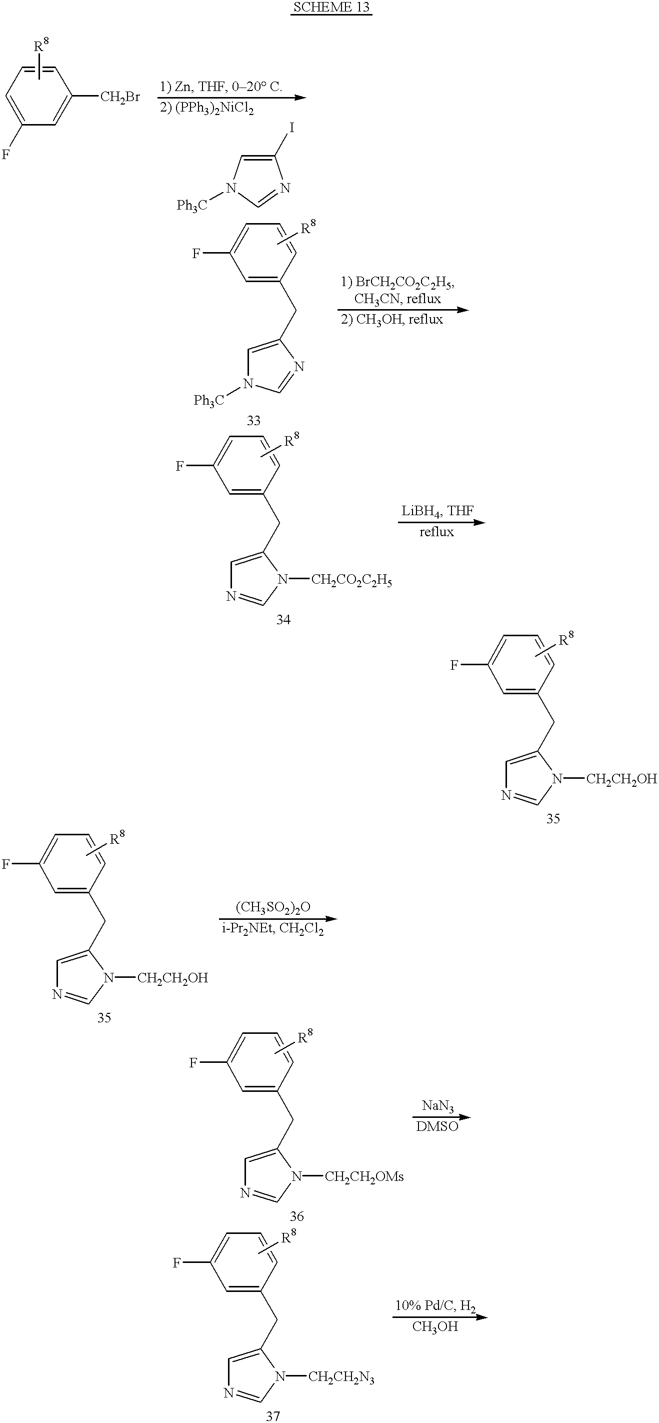

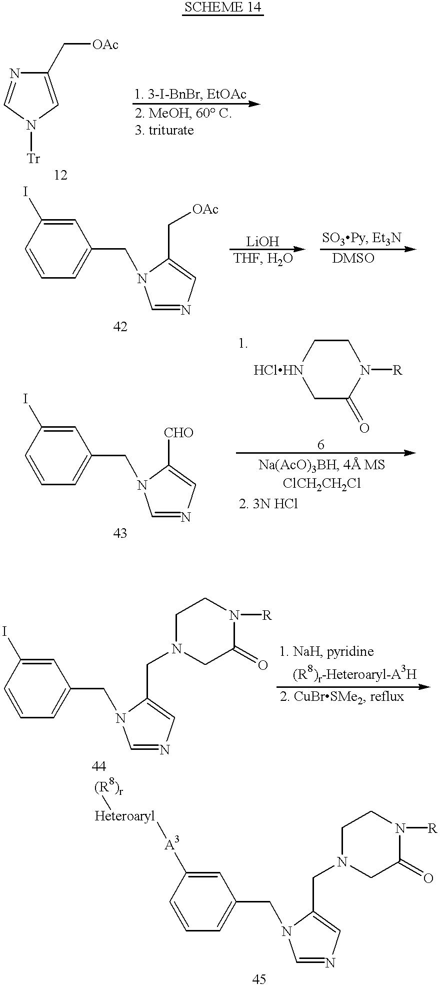

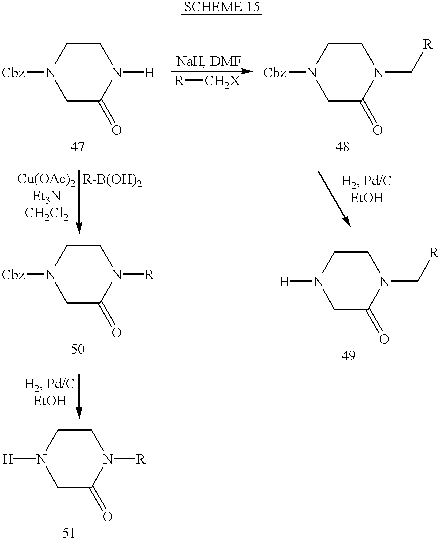

In a preferred embodiment of the instant invention the compounds of the invention are selective inhibitors of farnesyl-protein transferase. A compound is considered a selective inhibitor of famesyl-protein transferase, for example, when its in vitro farnesyl-protein transferase inhibitory activity, as assessed by the assay described in Example 3, is at least 100 times greater than the in vitro activity of the same compound against geranylgeranyl-protein transferase-type I in the assay described in Example 4. Preferably, a selective compound exhibits at least 1000 times greater activity against one of the enzymatic activities when comparing geranylgeranyl-protein transferase-type I inhibition and farnesyl-protein transferase inhibition.

It is also preferred that the selective inhibitor of famesyl-protein transferase is further characterized by:

a) an IC₅₀ (a measure of in vitro inhibitory activity) for inhibition of the prenylation of newly synthesized K-Ras protein more than about 100-fold higher than the EC₅₀ for the inhibition of the farnesylation of hDJ protein.

When measuring such IC₅₀s and EC₅₀s the assays described in Example 8 may be utilized.

It is also preferred that the selective inhibitor of farnesyl-protein transferase is further characterized by:

b) an IC₅₀ (a measurement of in vitro inhibitory activity) for inhibition of K4B-Ras dependent activation of MAP kinases in cells at least 100-fold greater than the EC₅₀ for inhibition of the famesylation of the protein hDJ in cells.

It is also preferred that the selective inhibitor of farnesyl-protein transferase is further characterized by:

c) an IC₅₀ (a measurement of in vitro inhibitory activity) against H-Ras dependent activation of MAP kinases in cells at least 1000 fold lower than the inhibitory activity (IC₅₀) against H-ras-CVLL (SEQ.ID.NO.: 1) dependent activation of MAP kinases in cells.

When measuring Ras dependent activation of MAP kinases in cells the assays described in Example 7 may be utilized.

In another preferred embodiment of the instant invention the compounds of the invention are dual inhibitors of farnesyl-protein transferase and geranylgeranyl-protein transferase type I. Such a dual inhibitor may be termed a Class II prenyl-protein transferase inhibitor and will exhibit certain characteristics when assessed in in vitro assays, which are dependent on the type of assay employed.

In a SEAP assay, such as described in Example 7, it is preferred that the dual inhibitor compound has an in vitro inhibitory activity (IC₅₀) that is less than about 12 μM against K4B-Ras dependent activation of MAP kinases in cells.

The Class II prenyl-protein transferase inhibitor may also be characterized by:

a) an IC₅₀ (a measurement of in vitro inhibitory activity) for inhibiting K4B-Ras dependent activation of MAP kinases in cells between 0.1 and 100 times the IC₅₀ for inhibiting the farnesylation of the protein hDJ in cells; and

b) an IC₅₀ (a measurement of in vitro inhibitory activity) for inhibiting K4B-Ras dependent activation of MAP kinases in cells greater than 5-fold lower than the inhibitory activity (IC₅₀) against expression of the SEAP protein in cells transfected with the pCMV-SEAP plasmid that constitutively expresses the SEAP protein.

The Class II prenyl-protein transferase inhibitor may also be characterized by:

a) an IC₅₀ (a measurement of in vitro inhibitory activity) against H-Ras dependent activation of MAP kinases in cells greater than 2 fold lower but less than 20,000 fold lower than the inhibitory activity (IC₅₀) against H-ras-CVLL (SEQ.ID.NO.: 1) dependent activation of MAP kinases in cells; and

b) an IC₅₀ (a measurement of in vitro inhibitory activity) against H-ras-CVLL dependent activation of MAP kinases in cells greater than 5-fold lower than the inhibitory activity (IC₅₀) against expression of the SEAP protein in cells transfected with the pCMV-SEAP plasmid that constitutively expresses the SEAP protein.

The Class II prenyl-protein transferase inhibitor may also be characterized by:

a) an IC₅₀ (a measurement of in vitro inhibitory activity) against H-Ras dependent activation of MAP kinases in cells greater than 10-fold lower but less than 2,500 fold lower than the inhibitory activity (IC₅₀) against H-ras-CVLL (SEQ.ID.NO.: 1) dependent activation of MAP kinases in cells; and

b) an IC₅₀ (a measurement of in vitro inhibitory activity) against H-ras-CVLL dependent activation of MAP kinases in cells greater than 5 fold lower than the inhibitory activity (IC₅₀) against expression of the SEAP protein in cells transfected with the pCMV-SEAP plasmid that constitutively expresses the SEAP protein.

A method for measuring the activity of the inhibitors of prenyl-protein transferase, as well as the instant combination compositions, utilized in the instant methods against Ras dependent activation of MAP kinases in cells is described in Example 7.

In yet another embodiment, a compound of the instant invention may be a more potent inhibitor of geranylgeranyl-protein transferase-type I than it is an inhibitor of farnesyl-protein transferase.

The instant compounds are useful as pharmaceutical agents for mammals, especially for humans. These compounds may be administered to patients for use in the treatment of cancer. Examples of the type of cancer which may be treated with the compounds of this invention include, but are not limited to, colorectal carcinoma, exocrine pancreatic carcinoma, myeloid leukemias and neurological tumors. Such tumors may arise by mutations in the ras genes themselves, mutations in the proteins that can regulate Ras activity (i.e., neurofibromin (NF-1), neu, src, abI, lck, fyn) or by other mechanisms.

The compounds of the instant invention inhibit farnesyl-protein transferase and the farnesylation of the oncogene protein Ras. The instant compounds may also inhibit tumor angiogenesis, thereby affecting the growth of tumors (J. Rak et al. Cancer Research, 55:4575-4580 (1995)). Such anti-angiogenesis properties of the instant compounds may also be useful in the treatment of certain forms of vision deficit related to retinal vascularization.

The compounds of this invention are also useful for inhibiting other proliferative diseases, both benign and malignant, wherein Ras proteins are aberrantly activated as a result of oncogenic mutation in other genes (i.e., the Ras gene itself is not activated by mutation to an oncogenic form) with said inhibition being accomplished by the administration of an effective amount of the compounds of the invention to a mammal in need of such treatment. For example, the composition is useful in the treatment of neurofibromatosis, which is a benign proliferative disorder.

The instant compounds may also be useful in the treatment of certain viral infections, in particular in the treatment of hepatitis delta and related viruses (J. S. Glenn et al. Science, 256:1331-1333 (1992).

The compounds of the instant invention are also useful in the prevention of restenosis after percutaneous transluminal coronary angioplasty by inhibiting neointimal formation (C. Indolfi et al. Nature medicine, 1:541-545(1995).

The instant compounds may also be useful in the treatment and prevention of polycystic kidney disease (D.L. Schaffner et al. American Journal of Pathology, 142:1051-1060 (1993) and B. Cowley, Jr. et al. FASEB Journal, 2:A3160 (1988)).

The instant compounds may also be useful for the treatment of fungal infections.

The instant compounds may also be useful as inhibitors of proliferation of vascular smooth muscle cells and therefore useful in the prevention and therapy of arteriosclerosis and diabetic vascular pathologies.

The compounds of the instant invention may also be useful in the prevention and treatment of endometriosis, uterine fibroids, dysfunctional uterine bleeding and endometrial hyperplasia.

In such methods of prevention and treatment as described herein, the prenyl-protein transferase inhibitors of the instant invention may also be co-administered with other well known therapeutic agents that are selected for their particular usefulness against the condition that is being treated. For example, the prenyl-protein transferase inhibitor may be useful in further combination with drugs known to supress the activity of the ovaries and slow the growth of the endometrial tissue. Such drugs include but are not limited to oral contraceptives, progestins, danazol and GnRH (gonadotropin-releasing hormone) agonists.

Administration of the prenyl-protein transferase inhibitor may also be combined with surgical treatment of endometriosis (such as surgical removal of misplaced endometrial tissue) where appropriate.

The instant compounds may also be useful as inhibitors of corneal inflammation. These compounds may improve the treatment of corneal opacity which results from cauterization-induced corneal inflammation. The instant compounds may also be useful in reducing corneal edema and neovascularization. (K. Sonoda et al., Invest. Ophthalmol. Vis. Sci., 1998, vol. 39, p 2245-2251).

The compounds of this invention may be administered to mammals, preferably humans, either alone or, preferably, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

Additionally, the compounds of the instant invention may be administered to a mammal in need thereof using a gel extrusion mechanism (GEM) device, such as that described in U.S. Ser. No. 60/144,643, filed on Jul. 20, 1999, which is hereby incorporated by reference. The compounds of the instant invention may also be administered to a mammal in need thereof using an osmotic controlled release drug delivery device, such as those described in U.S. Ser. No. 60/162,589 and U.S. Ser. No. 60/162,719, co-filed on Oct. 29, 1999, and herein incorporated by reference.

As used herein, the term “composition” is intended to encompass a product comprising the specified ingredients in the specific amounts, as well as any product which results, directly or indirectly, from combination of the specific ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula A-1 may also be administered in the form of a suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula A-1 are employed. (For purposes of this application, topical application shall include mouth washes and gargles.) The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

The compounds of the instant invention may also be co-administered with other well known therapeutic agents that are selected for their particular usefulness against the condition that is being treated. For example, the compounds of the instant invention may also be co-administered with other well known cancer therapeutic agents that are selected for their particular usefulness against the condition that is being treated. Included in such combinations of therapeutic agents are combinations of the instant farnesyl-protein transferase inhibitors and an antineoplastic agent. It is also understood that such a combination of antineoplastic agent and inhibitor of farnesyl-protein transferase may be used in conjunction with other methods of treating cancer and/or tumors, including radiation therapy and surgery. It is further understood that any of the therapeutic agents described herein may also be used in combination with a compound of the instant invention and an antineoplastic agent.

Examples of an antineoplastic agent include, in general, microtubule-stabilizing agents (such as paclitaxel (also known as Taxol®), docetaxel (also known as Taxotere®), epothilone A, epothilone B, desoxyepothilone A, desoxyepothilone B or their derivatives); microtubule-disruptor agents; alkylating agents, for example, nitrogen mustards, ethyleneimine compounds, alkyl sulfonates and other compounds with an alkylating action such as nitrosoureas, cisplatin, and dacarbazine; anti-metabolites, for example, folic acid, purine or pyrimidine antagonists; epidophyllotoxin; an antineoplastic enzyme; a topoisomerase inhibitor; procarbazine; mitoxantrone; platinum coordination complexes; biological response modifiers and growth inhibitors; mitotic inhibitors, for example, vinca alkaloids and derivatives of podophyllotoxin; cytotoxic antibiotics; hormonal/anti-hormonal therapeutic agents, haematopoietic growth factors and antibodies (such as trastuzumab (HerceptinTM)).

Example classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, carminomycin, daunorubicin, aminopterin, methotrexate, methopterin, dichloro-methotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, dactinomycin, mechlorethamine (nitrogen mustard), streptozocin, cyclophosphamide, carmustine (BCNU), lomustine (CCNU), procarbazine, mitomycin, cytarabine, etoposide, methotrexate, bleomycin, chlorambucil, camptothecin, CPT-11, topotecan, ara-C, bicalutamide, flutamide, leuprolide, pyridobenzoindole derivatives, interferons and interleukins. Particular examples of antineoplastic, or chemotherapeutic, agents are described, for example, by D. J. Stewart in “Nausea and Vomiting: Recent Research and Clinical Advances”, Eds. J. Kucharczyk, et al., CRC Press Inc., Boca Raton, Florida, USA (1991), pages 177-203, especially page 188. See also, R. J. Gralla, et al., Cancer Treatment Reports, 68(1), 163-172 (1984).

The preferred class of antineoplastic agents is the taxanes and the preferred antineoplastic agent is paclitaxel.

The compounds of the instant invention may also be co-administered with antisense oligonucleotides which are specifically hybridizable with RNA or DNA deriving from human ras gene. Such antisense oligonucleotides are described in U.S. Pat. No. 5,576,208 and PCT Publication No. WO 99/22772. The instant compounds are particularly usefule when co-administered with the antisense oligonucleotide comprising the amino acid sequence of SEQ.ID.NO: 2 of U.S. Pat. No. 5,576,208.

Certain compounds of the instant invention may exhibit very low plasma concentrations and significant inter-individual variation in the plasma levels of the compound. It is believed that very low plasma concentrations and high intersubject variability achieved following administration of certain prenyl-protein transferase inhibitors to mammals may be due to extensive metabolism by cytochrome P450 enzymes prior to entry of drug into the systemic circulation. Prenyl-protein transferase inhibitors may be metabolized by cytochrome P450 enzyme systems, such as CYP3A4, CYP2D6, CYP2C₉, CYP2C₁₉ or other cytochrome P450 isoform. If a compound of the instant invention demonstrates an affinity for one or more of the cytochrome P450 enzyme systems, another compound with a higher affinity for the P450 enzyme(s) involved in metabolism should be administered concomitantly. Examples of compounds that have a comparatively very high affinity for CYP3A4, CYP2D6, CYP2C₉, CYP2C₁₉ or other P450 isoform include, but are not limited to, piperonyl butoxide, troleandomycin, erythromycin, proadifen, isoniazid, allylisopropylacetamide, ethinylestradiol, chloramphenicol, 2-ethynylnaphthalene and the like. Such a high affinity compound, when employed in combination with a compound of formula A-1, may reduce the inter-individual variation and increase the plasma concentration of a compound of formula A-I to a level having substantial therapeutic activity by inhibiting the metabolism of the compound of formula A-1. Additionally, inhibiting the metabolism of a compound of the instant invention prolongs the pharmacokinetic half-life, and thus the pharmacodynamic effect, of the compound.

A compound of the present invention may be employed in conjunction with antiemetic agents to treat nausea or emesis, including acute, delayed, late-phase, and anticipatory emesis, which may result from the use of a compound of the present invention, alone or with radiation therapy. For the prevention or treatment of emesis a compound of the present invention may be used in conjunction with other anti-emetic agents, especially neurokinin-1 receptor antagonists, SHT3 receptor antagonists, such as ondansetron, granisetron, tropisetron, and zatisetron, GABAB receptor agonists, such as baclofen, or a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712. For the treatment or prevention of emesis, conjunctive therapy with a neurokinin-1 receptor antagonist, a 5HT3 receptor antagonist and a corticosteroid is preferred.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications.

A particularly preferred neurokinin-1 receptor antagonist for use in conjunction with the compounds of the present invention is 2-(R)-(1-(R)-(3,5-bis (trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

For the treatment of cancer, it may be desirable to employ a compound of the present invention in conjunction with another pharmacologically active agent(s). A compound of the present invention and the other pharmacologically active agent(s) may be administered to a patient simultaneously, sequentially or in combination. For example, the present compound may employed directly in combination with the other active agent(s), or it may be administered prior, concurrent or subsequent to the administration of the other active agent(s). In general, the currently available dosage forms of the known therapeutic agents for use in such combinations will be suitable.

For example, a compound of the present invention may be presented together with another therapeutic agent in a combined preparation, such as with an antiemetic agent for simultaneous, separate, or sequential use in the relief of emesis associated with employing a compound of the present invention and radiation therapy. Such combined preparations may be, for example, in the form of a twin pack. A preferred combination comprises a compound of the present invention with antiemetic agents, as described above.

Radiation therapy, including x-rays or gamma rays which are delivered from either an externally applied beam or by implantation of tiny radioactive sources, may also be used in combination with the instant inhibitor of prenyl-protein transferase alone to treat cancer.

Additionally, compounds of the instant invention may also be useful as radiation sensitizers, as described in WO 97/38697, published on Oct. 23, 1997, and herein incorporated by reference.

The instant compounds may also be useful in combination with other inhibitors of parts of the signaling pathway that links cell surface growth factor receptors to nuclear signals initiating cellular proliferation. Thus, the instant compounds may be utilized in combination with farnesyl pyrophosphate competitive inhibitors of the activity of farnesyl-protein transferase or in combination with a compound which has Raf antagonist activity. The instant compounds may also be co-administered with compounds that are selective inhibitors of geranylgeranyl protein transferase.

In particular, if the compound of the instant invention is a selective inhibitor of farnesyl-protein transferase, co-administration with a compound(s) that is a selective inhibitor of geranylgeranyl protein transferase may provide an improved therapeutic effect.

In particular, the compounds disclosed in the following patents and publications may be useful as farnesyl pyrophosphate-competitive inhibitor component of the instant composition: U.S. Ser. Nos. 08/254,228 and 08/435,047. Those patents and publications are incorporated herein by reference.

In practicing methods of this invention, which comprise administering, simultaneously or sequentially or in any order, two or more of a protein substrate-competitive inhibitor and a farnesyl pyrophosphate-competitive inhibitor, such administration can be orally or parenterally, including intravenous, intramuscular, intraperitoneal, subcutaneous, rectal and topical routes of administration. It is preferred that such administration be orally. It is more preferred that such administration be orally and simultaneously. When the protein substrate-competitive inhibitor and farnesyl pyrophosphate-competitive inhibitor are administered sequentially, the administration of each can be by the same method or by different methods.

The instant compounds may also be useful in combination with an integrin antagonist for the treatment of cancer, as described in U.S. Ser. No. 09/055,487, filed Apr. 6, 1998, and WO 98/44797, published on Oct. 15, 1998, which are incorporated herein by reference.

As used herein the term an integrin antagonist refers to compounds which selectively antagonize, inhibit or counteract binding of a physiological ligand to an integrin(s) that is involved in the regulation of angiogenisis, or in the growth and invasiveness of tumor cells. In particular, the term refers to compounds which selectively antagonize, inhibit or counteract binding of a physiological ligand to the avp3 integrin, which selectively antagonize, inhibit or counteract binding of a physiological ligand to the αvβ5 integrin, which antagonize, inhibit or counteract binding of a physiological ligand to both the αvβ3 integrin and the αvβ5 integrin, or which antagonize, inhibit or counteract the activity of the particular integrin(s) expressed on capillary endothelial cells. The term also refers to antagonists of the α1β1, α2β1, α5β1, α6β1 and α6β4 integrins. The term also refers to antagonists of any combination of αvβ3 integrin, αvβ5 integrin, α1β1, α2β1, α5β1, α6β1 and α6β4 integrins. The instant compounds may also be useful with other agents that inhibit angiogenisis and thereby inhibit the growth and invasiveness of tumor cells, including, but not limited to angiostatin and endostatin.

The instant compounds may also be useful in combination with an inhibitor of 3-hydroxy-3-methylglutaryl-CoA reductase (HMG-COA reductase) for the treatment of cancer. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of IMG-CoA reductase” have the same meaning when used herein.

Examples of HMG-CoA reductase inhibitors that may be used include but are not limited to lovastatin (MEVACOR®; see U.S. Pat. Nos. 4,231,938; 4,294,926; 4,319,039), simvastatin (ZOCOR®; see U.S. Pat. Nos. 4,444,784; 4,820,850; 4,916,239), pravastatin (PRAVACHOL®; see U.S. Pat. Nos. 4,346,227; 4,537,859; 4,410,629; 5,030,447 and 5,180,589), fluvastatin (LESCOL®; see U.S. Pat. Nos. 5,354,772; 4,911,165; 4,929,437; 5,189,164; 5,118,853; 5,290,946; 5,356,896), atorvastatin (LIPITOR®; see U.S. Pat. Nos. 5,273,995; 4,681,893; 5,489,691; 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (Feb. 5, 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

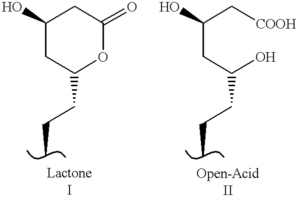

In HMG-CoA reductase inhibitors, where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, omithine, choline, N,N′-dibenzylethylenedi amine, chloroprocaine, diethanol amine, procaine, N-benzylphenethylamine, 1-p-chlorobenzyl-2-pyrrolidine-1′-yl-methylbenzimidazole, diethylamine, piperazine, an d tris(hydroxymethyl)-aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

Similarly, the instant compounds may be useful in combination with agents that are effective in the treatment and prevention of NF-1, restenosis, polycystic kidney disease, infections of hepatitis delta and related viruses and fungal infections.

If formulated as a fixed dose, such combination products employ the combinations of this invention within the dosage range described above and the other pharmaceutically active agent(s) within its approved dosage range. Combinations of the instant invention may alternatively be used sequentially with known pharmaceutically acceptable agent(s) when a multiple combination formulation is inappropriate.

The instant compounds may also be useful in combination with prodrugs of antineoplastic agents. In particular, the instant compounds may be co-administered either concurrently or sequentially with a conjugate (termed a “PSA conjugate”) which comprises an oligopeptide, that is selectively cleaved by enzymatically active prostate specific antigen (PSA), and an antineoplastic agent. Such co-administration will be particularly useful in the treatment of prostate cancer or other cancers which are characterized by the presence of enzymatically active PSA in the immediate surrounding cancer cells, which is secreted by the cancer cells.

Compounds which are PSA conjugates and are therefore useful in such a co-administration, and methods of synthesis thereof, can be found in the following patents, pending patent applications and publications which are herein incorporated by reference:

U.S. Pat. No. 5,599,686, granted on Feb. 4, 1997;

WO 96/00503 (Jan. 11, 1996); USSN 08/404,833, filed on Mar. 15, 1995;

U.S. Ser. No. 08/468,161, filed on Jun. 6, 1995;

U.S. Pat. No. 5,866,679, granted on Feb. 2, 1999;

WO 98/10651 (Mar. 19, 1998); U.S. Ser. No. 08/926,412, filed on Sep. 9, 1997;

WO 98/18493 (May 7, 1998); U.S. Ser. No. 08/950,805, filed on Oct. 14, 1997;

WO 99/02175 (Jan. 21, 1999); U.S. Ser. No. 09/112,656, filed on Jul. 9, 1998; and

WO 99/28345 (Jun. 10, 1999); U.S. Ser. No. 09/193,365, filed on Nov. 17, 1998.

Compounds which are described as prodrugs wherein the active therapeutic agent is released by the action of enzymatically active PSA and therefore may be useful in such a co-administration, and methods of synthesis thereof, can be found in the following patents, pending patent applications and publications, which are herein incorporated by reference: WO 98/52966 (Nov. 26, 1998).

All patents, publications and pending patent applications identified are herein incorporated by reference.

The compounds of the instant invention are also useful as a component in an assay to rapidly determine the presence and quantity of famesyl-protein transferase (FPTase) in a composition. Thus the composition to be tested may be divided and the two portions contacted with mixtures which comprise a known substrate of FPTase (for example a tetrapeptide having a cysteine at the amine terminus) and farnesyl pyrophosphate and, in one of the mixtures, a compound of the instant invention. After the assay mixtures are incubated for a sufficient period of time, well known in the art, to allow the FPTase to farnesylate the substrate, the chemical content of the assay mixtures may be determined by well known immuno-logical, radiochemical or chromatographic techniques. Because the compounds of the instant invention are selective inhibitors of FPTase, absence or quantitative reduction of the amount of substrate in the assay mixture without the compound of the instant invention relative to the presence of the unchanged substrate in the assay containing the instant compound is indicative of the presence of FPTase in the composition to be tested.

It would be readily apparent to one of ordinary skill in the art that such an assay as described above would be useful in identifying tissue samples which contain farnesyl-protein transferase and quantitating the enzyme. Thus, potent inhibitor compounds of the instant invention may be used in an active site titration assay to determine the quantity of enzyme in the sample. A series of samples composed of aliquots of a tissue extract containing an unknown amount of farnesyl-protein transferase, an excess amount of a known substrate of FPTase (for example a tetrapeptide having a cysteine at the amine terminus) and farnesyl pyrophosphate are incubated for an appropriate period of time in the presence of varying concentrations of a compound of the instant invention. The concentration of a sufficiently potent inhibitor (i.e., one that has a Ki substantially smaller than the concentration of enzyme in the assay vessel) required to inhibit the enzymatic activity of the sample by 50% is approximately equal to half of the concentration of the enzyme in that particular sample.

### EXAMPLES

Examples provided are intended to assist in a further understanding of the invention. Particular materials employed, species and conditions are intended to be further illustrative of the invention and are not intended to limit the reasonable scope thereof.

### Example 1

Preparation of 1-(3-chlorophenyl)-4-[1-(3-(3-pyridyloxy)-4-cyanobenzyl)-5-imidazolylmethyl]-2-piperazinone dihydrochloride

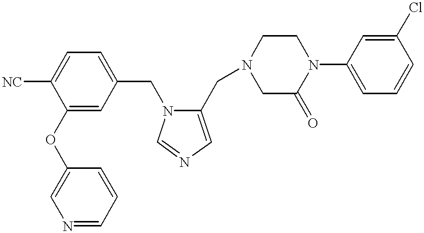

Step A: Preparation of 1-triphenylmethyl-4-(hydroxymethyl)-imidazole

To a solution of 4-(hydroxymethyl)imidazole hydrochloride (35.0 g, 260 mmol) in 250 mL of dry DMF at room temperature was added triethylamine (90.6 mL, 650 mmol). A white solid precipitated from the solution. Chlorotriphenylmethane (76.1 g, 273 mmol) in 500 mL of DMF was added dropwise. The reaction mixture was stirred for 20 hours, poured over ice, filtered, and washed with ice water. The resulting product was slurried with cold dioxane, filtered, and dried in vacuo to provide the titled product as a white solid.

Step B: Preparation of 1-triphenylmethyl-4-(acetoxymethyl)-imidazole

An alcohol, as described in Step A, (260 mmol, prepared above) was suspended in 500 mL of pyridine. Acetic anhydride (74 mL, 780 mmol) was added dropwise, and the reaction was stirred for 48 hours during which it became homogeneous. The solution was poured into 2 L of EtOAc, washed with water (3×1 L), 5% aq. HCl soln. (2×1 L), sat. aq. NaHCO₃, and brine, then dried (Na₂SO₄), filtered, and concentrated in vacuo to provide the crude product. The acetate was isolated as a white powder.

Step C: Preparation of 4-cyano-3-fluorotoluene

To a degassed solution of 4-bromo-3-fluorotoluene (50.0 g, 264 mmol) in 500 mL of DMF was added Zn(CN)₂ (18.6 g, 159 mmol) and Pd(PPh₃)₄ (6.1 g, 5.3 mmol). The reaction was stirred at 80° C. for 6 hours, then cooled to room temperature. The solution was poured into EtOAc, washed with water, sat. aq. NaHCO₃, and brine, then dried (Na₂SO₄), filtered, and concentrated in vacuo to provide the crude product. Purification by silica gel chromatography (0-5% EtOAc/hexane) provided the titled product.

Step D: Preparation of 4-cyano-3-fluorobenzylbromide

To a solution of the product described in Step C (22.2 g, 165 mmol) in 220 mL of carbontetrachloride was added N-bromosuccinimide (29.2 g, 164 mmol) and benzoylperoxide (1.1 g). The reaction was heated to reflux for 30 minutes, then cooled to room temperature. The solution was concentrated in vacuo to one-third the original volume, poured into EtOAc, washed with water, sat. aq. NaHCO₃, and brine, then dried (Na₂SO₄), filtered, and concentrated in vacuo to provide the crude product. Analysis by 1H NMR indicated only partial conversion, so the crude material was subjected to the same reaction conditions for 2.5 hours, using 18 g (102 mmol) of N-bromosuccinimide. After workup, the crude material was purified by silica gel chromatography (0-10% EtOAc/hexane) to provide the desired product.

Step E: Preparation of 1-(4-cyano-3-fluorobenzyl)-5-(acetoxymethyl)-imidazole hydrobromide

A solution of the product described in Step B (36.72 g, 96.14 mmol) and the product described in Step D (20.67 g, 96.14 mmol) in 250 mL of EtOAc was stirred at 60° C. for 20 hours, during which a white precipitate formed. The reaction was cooled to room temperature and filtered to provide the solid imidazolium bromide salt. The filtrate was concentrated in vacuo to a volume of 100 mL, reheated at 60° C. for two hours, cooled to room temperature, and filtered again. The filtrate was concentrated in vacuo to a volume of 40 mL, reheated at 60° C. for another two hours, cooled to room temperature, and concentrated in vacuo to provide a pale yellow solid. All of the solid material was combined, dissolved in 300 mL of methanol, and warmed to 60° C. After two hours, the solution was reconcentrated in vacuo to provide a white solid which was triturated with hexane to remove soluble materials. Removal of residual solvents in vacuo provided the titled product hydrobromide as a white solid.

Step F: Preparation of 1-(4-cyano-3-fluorobenzyl)-5-(hydroxymethyl) imidazole

To a solution of the product described in Step E (31.87 g, 89.77 mmol) in 300 mL of 2:1 THF/water at 0° C. was added lithium hydroxide monohydrate (7.53 g, 179 mmol). After two hours, the reaction was concentrated in vacuo to a 100 mL volume, stored at 0° C. for 30 minutes, then filtered and washed with 700 mL of cold water to provide a brown solid. This material was dried in vacuo next to P₂O₅ to provide the titled product as a pale brown powder.

Step G: Preparation of 1-(4-cyano-3-fluorobenzyl)-5-imidazolecarboxaldehyde

To a solution of the alcohol described in Step F (2.31 g, 10.0 mmol) in 20 mL of DMSO at 0° C. was added triethylamine (5.6 mL, 40 mmol), then SO₃-pyridine complex (3.89 g, 25 mmol). After 30 minutes, the reaction was poured into EtOAc, washed with water and brine, dried (Na₂SO₄), filtered, and concentrated in vacuo to provide the aldehyde as a pale yellow powder.

Step H: Preparation of N-(3-chlorophenyl)ethylenediamine hydrochloride

To a solution of 3-chloroaniline (30.0 mL, 284 mmol) in 500 mL of dichloromethane at 0° C. was added dropwise a solution of 4 N HCl in 1,4-dioxane (80 mL, 320 mmol HCl). The solution was warmed to room temperature, then concentrated to dryness in vacuo to provide a white powder. A mixture of this powder with 2-oxazolidinone (24.6 g, 282 mmol) was heated under nitrogen atmosphere at 160° C. for 10 hours, during which the solids melted, and gas evolution was observed. The reaction was allowed to cool, forming the crude diamine hydrochloride salt as a pale brown solid.

Step I: Preparation of N-(tert-butoxycarbonyl)-N′-(3-chlorophenyl) ethylenediamine

The amine hydrochloride described in Step H (ca. 282 mmol, crude material prepared above) was taken up in 500 mL of THF and 500 mL of sat. aq. NaHCO₃ soln., cooled to 0° C., and di-tert-butylpyrocarbonate (61.6 g, 282 mmol) was added. After 30 h, the reaction was poured into EtOAc, washed with water and brine, dried (Na₂SO₄), filtered, and concentrated in vacuo to provide the titled carbamate as a brown oil.

Step J: Preparation of N-[2-(tert-butoxycarbamoyl)ethyl]-N-(3-chlorophenyl)-2-chloroacetamide

A solution of the product described in Step 1 (77 g, ca. 282 mmol) and triethylamine (67 mL, 480 mmol) in 500 mL of CH₂Cl₂ was cooled to 0° C. Chloro-acetyl chloride (25.5 mL, 320 mmol) was added dropwise, and the reaction was maintained at 0° C. with stirring. After 3 h, another portion of chloroacetyl chloride (3.0 mL) was added dropwise. After 30 min, the reaction was poured into EtOAc (2 L) and washed with water, sat. aq. NH₄Cl soln, sat. aq. NaHCO₃ soln., and brine. The solution was dried (Na₂SO₄), filtered, and concentrated in vacuo to provide the chloroacetamide as a brown oil.

Step K: Preparation of 4-(tert-butoxycarbonyl)-1-(3-chlorophenyl)-2-piperazinone

To a solution of the chloroacetamide described in Step J (ca. 282 mmol) in 700 mL of dry DMF was added K2CO₃ (88 g, 0.64 mol). The solution was heated in an oil bath at 70-75° C. for 20 hours, cooled to room temperature, and concentrated in vacuo to remove ca. 500 mL of DMF. The remaining material was poured into 33% EtOAc/hexane, washed with water and brine, dried (Na₂SO₄), filtered, and concentrated in vacuo to provide the product as a brown oil. This material was purified by silica gel chromatography (25-50% EtOAc/hexane) to yield pure product, along with a sample of product (ca. 65% pure by HPLC) containing a less polar impurity.

Step L: Preparation of 1-(3-chlorophenyl)-2-piperazinone hydrochloride

Through a solution of the product described in Step K (5.30 g, 17.1 mmol) in 60 mL of ethyl acetate at 0° C. was bubbled anhydrous HCl gas for 5 minutes. After 15 minutes, the solution was concentrated in vacuo to provide the titled salt as a white foam.

Step M: Preparation of 1-(3-chlorophenyl)-4-[1-(4-cyano-3-fluorobenzyl)-5-imidazolylmethyl]-2-piperazinone

To a solution of the amine hydrochloride from Step L (1.36 g, 5.5 mmol) and the aldehyde from Step G (1.26 g, 5.5 mmol) in 20 mL of 1,2-dichloroethane at 0° C. was added 4A powdered molecular sieves (2 g), followed by sodium triacetoxyborohydride (1.75 g, 8.3 mmol). The reaction was stirred at OC for 30 minutes, then warmed to room temperature. After 4 hours, the reaction was poured into EtOAc, washed with dilute aq. NaHCO₃ and brine, dried (Na₂SO₄), filtered, and concentrated in vacuo. The resulting product was taken up in CH₂Cl₂, and propylamine was added. The mixture was stirred for 30 minutes, then concentrated in vacuo. This material was purified by silica gel chromatography (50-70% acetone/CH₂Cl₂) to give the titled product as a white solid.

Step N: Preparation of 1-(3-chlorophenyl)-4-[1-(3-(3-pyridyloxy)-4-cyanobenzyl)-5-imidazolylmethyl]-2-piperazinone (1), dihydrochloride

To a solution of the product described in Step M (61 mg, 0.14 mmol) in 2 mL of DMSO was added cesium carbonate (140 mg, 0.43 mmol) and 3-hydroxy-pyridine (41 mg, 0.43 mmol). The reaction was stirred at room temperature overnight under argon. The solution was poured into EtOAc and washed with water and brine, dried (Na₂SO₄), filtered, and concentrated in vacuo. The resulting product was purified on 1 mm silica gel preparative TLC plates (10% MeOH/CHCl3), taken up in CH₂Cl₂ and treated with excess 1 M HCl/ether solution, and concentrated in vacuo to provide the titled product dihydrochloride as a white powder.

FAB mass spectrum m/e 499 (M+1).

Analysis calculated for C₂₇H₂₃ClN₆O₂.2.00 HCl.0.90 H₂O.0.55 CH₂Cl₂: C, 52.12; H, 4.43; N, 13.24;

Found: C, 52.16; H, 4.44; N, 12.99.

### Example 2

Preparation of 1-(2-(n-Butyloxy)phenyl)-4-[1-(3-((6-methyl-2-pyridyl)oxy)-4-cyanobenzyl)-2-methyl-5-imidazolylmethyl]-2-piperazinone dihydrochloride

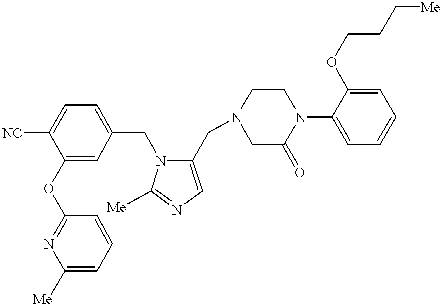

Step A: Preparation of 1-(4-cyano-3-fluorobenzyl)-2-methyl-5-imidazolecarboxaldehyde

To a solution of a bromide, as described in Step D of Example 1, (1.26 g, 5.9 mmol) in 10 mL of DMF at 0° C. was added 4-formyl-2-methylimidazole (0.650 g, 5.9 mmol) and cesium carbonate (2.9 g, 8.9 mmol). After 2 hours, the reaction was poured into 2:1 EtOAc:hexane, washed with water and brine, dried (Na₂SO₄), filtered, and concentrated in vacuo to provide the crude product mixture. The material was purified by silica gel chromatography (2-5% MeOH/CHCl₃) to provide the titled product along with the regioisomer 1-(4-cyano-3-fluorobenzyl)-2-methyl-4-imidazolecarboxaldehyde and a mixed fraction.

Step B: Preparation 2-[(3,4-dichlorobenzyl)oxy]nitrobenzene

A solution of 3,4-dichlorobenzyl alcohol (25.0 g, 141 mmol), 2-fluorobenzaldehyde (14.9 mL, 141 mmol) and potassium carbonate (39.0 g, 282 mmol) in 100 mL of dry DMF was stirred a 60° C. overnight. The DMF was removed in vacuo, and the resulting product was taken up in EtOAc/water. The organic phase was washed with brine, dried (Na₂SO₄), filtered, and concentrated in vacuo to provide the titled compound.

Step C: Preparation 2-[(3,4-dichlorobenzvl)oxy]aniline hydrochloride

A solution of the product described in Step B (39.5 g, 132 mmol), iron filings (26 g, 462 mmol) and acetic acid (57 mL) in 250 mL of methanol was heated to reflux. After 3.5 hours, the solution was cooled, filtered and the filter cake was washed with methanol. The filtrate was concentrated in vacuo, taken up in EtOAc, and washed with sat. NaHCO₃ solution and brine. The resulting solution was dried with sodium sulfate, filtered, and concentrated in vacuo to provide the aniline product. This was dissolved in 100 mL methylene choloride, and dry HCl gas was bubbled through the solution at 0° C. Concentration in vacuo provided the titled compound.

Step D: Preparation of N-[2-((3,4-dichlorobenzyl)-oxy)phenyl]ethylenediamine

A solution of the aniline hydrochloride described in Step C (30.0 g, 98.5 mmol) and 2-oxazolidinone (8.6 g, 98.5 mmol) in 30 niL of 2-(2-methoxy-ethoxy)ethanol was heated to 160° C. for 3.5 hours, during which gas evolution was observed. The reaction was cooled, then filtered, then partitioned between EtOAc and aqueous NaHCO₃. After washing with brine, the solution was concentrated in vacuo. The resulting product was purified by silica gel chromatography (95:5:0.5-90:10:1; CHCl_{3/}MeOH/NH₄OH) to provide the titled compound.

Step E: Preparation of N-(tert-butoxycarbonyl)-N′[2-((3,4-dichlorobenzyl)-oxy)phenyl]ethylenediamine

The product described in Step D (20.8 g, 66.8 mmol) was taken up in 50 mL of TiHF and 50 mL of sat. aq. NaHCO₃ soln., and cooled to 0° C. Di-tert-butylpyrocarbonate (14.6 g, 66.8 mmol) was added, and the solution was allowed to warm to room temperature. After 3.5 h, the reaction was poured into EtOAc, washed with water and brine, dried (Na₂SO₄), filtered, and concentrated in vacuo to provide the titled carbamate.

Step F: Preparation of N-[2-(tert-butoxycarbamoyl)ethyl]-N′[2-((3,4-dichlorobenzvl)-oxy)phenyl]-2-chloroacetamide

The product described in Step E (20.3 g, 49.4 mmol) was taken up in 150 mL of THF and 100 mL of sat. aq. NaHCO₃ soln., and cooled to 0° C. Chloro-acetylchloride (4.4 mL, 54.4 mmol) was added dropwise, and the solution was stirred for two hours. Another 100 mL of sat NaHCO₃ and 50 mL EtOAc were added, followed by an additional portion of chloroacetylchloride (1.0 mL). After 1.5 h, the reaction was poured into EtOAc, washed with water and brine, dried (Na₂SO₄), filtered, and concentrated in vacuo to provide the crude solid product, which was re-precipitated from ether/hexane and filtered to give the titled compound.

Step G: Preparation of 4-(tert-butoxycarbonyl)-1-[2-((3,4-dichlorobenzyl)-oxy)phenyl]-2-piperazinone

To a solution of the chloroacetamide described in Step F (12.4 g, 25.4 mmol) in 75 mL of dry DMF was added Cs2CO₃ (24.4 g, 75 mmol). The solution was heated in an oil bath at 45° C. for 3.5 hours, cooled to room temperature, poured into EtOAc/water. The organic phase was washed with water and brine, dried (Na₂SO₄), filtered, and concentrated in vacuo to provide the titled product.

Step H: Preparation of 4-(tert-Butoxycarbonyl)-1-(2-hydroxyphenyl)-2-piperazinone

To a solution of the piperazinone described in Step G (2.00 g, 4.43 mmol) in 25 mL of methylene chloride was added sodium iodide (2.0 g, 13.3 mmol), and the solution was cooled to −15° C. Solid AlBr₃ was added (2.4 g, 8.9 mmol), and the solution was allowed to warm to room temperature and stir overnight. The reaction was diluted with 25 mL methylene chloride and 50 mL sat. NaHCO₃ solution, and di-tert-butylpyrocarbonate (1.95 g, 8.9 mmol) was added at room temperature.

After 5 hours, the layers were separated, the aqueous phase was extracted with EtOAc, and the combined organics were dried (Na₂SO₄), filtered, and concentrated in vacuo. The resulting product was purified by silica gel chromatography (25-100% EtOAc/hexane) to provide the titled compound.

Step I: Preparation of 4-(tert-Butoxycarbonyl)-1-[2-((n-butyl)oxy)phenyl]-2-piperazinone

To a solution of the phenol described in Step H (200 mg, 0.68 mmol) in 5 mL of dry DMF was added iodobutane (0.085 mL, 0.75 mmol) and Cs₂CO₃ (443 mg, 1.36 mmol). The reaction was stirred at room temperature overnight, then poured into EtOAc and washed with water, sat. NaHCO₃, and brine. The solution was dried (Na₂SO₄), filtered, and concentrated in vacuo to provide the titled product.

Step J: Preparation of 1-[2-((n-Butyl)oxy)phenyl]-2-piperazinone hydrochloride

Through a solution of the product described in Step 1 (233 mg, 0.67 mmol) in 10 mL of ethyl acetate at 0° C. was bubbled anhydrous HCl gas for 5 minutes. After 30 minutes, the solution was concentrated in vacuo to provide the titled salt as a white foam.

Step K: Preparation of 1-[2-((n-Butyl)oxy)phenyl]-4-[1-(4-cyano-3-fluorobenzvl)-2-methyl-5-imidazolylmethyl]-2-piperazinone

To a solution of the amine hydrochloride described in Step J (181 mg, 0.64 mmol) and the aldehyde described in Step A (170 mg, 0.70 mmol) in 5 mL of 1,2-dichloroethane was added 4A powdered molecular sieves (0.5 g), followed by sodium triacetoxyborohydride (203 mg, 0.96 mmol). The reaction was stirred at room temperature overnight. The reaction was poured into EtOAc, washed with dilute aq. NaHCO₃ and brine, dried (Na₂SO₄), filtered, and concentrated in vacuo. This material was purified by silica gel chromatography (2-5% MeOH/CHCl₃) to give the titled product as a white solid.

Step L: Preparation of 1-(2-(n-Butyloxy)phenyl)-4-[1-(3-((6-methyl-2-pyridyl)oxy)-4-cyanobenzyl)-2-methyl-5-imidazolylmethyl]-2-piperazinone) dihydrochloride

The titled product was prepared from the product described in Step K (112 mg, 0.24 mmol) using the procedure described in Example 1 Step N, except that 2-hydroxy-6-methylpyridine was used instead of 3-hydroxypyridine. The titled dihydrochloride was isolated as a white solid.

ES mass spectrum m/e 565.3 (M+1).

### Example 3

In vitro Inhibition of Ras Farnesyl Transferase

Transferase Assays. Isoprenyl-protein transferase activity assays are carried out at 30° C. unless noted otherwise. A typical reaction contains (in a final volume of 50 μL): [³H]farnesyl diphosphate, Ras protein, 50 mM HEPES, pH 7.5, 5 mM MgCL₂, 5 mM dithiothreitol, 10 μM ZnCL₂, 0.1% polyethyleneglycol (PEG) (15,000-20,000 mw) and isoprenyl-protein transferase. The FPTase employed in the assay is prepared by recombinant expression as described in Omer, C. A., Kral, A. M., Diehl, R. E., Prendergast, G. C., Powers, S., Allen, C. M., Gibbs, J. B. and Kohl, N. E. (1993) Biochemistry 32:5167-5176. After thermally pre-equilibrating the assay mixture in the absence of enzyme, reactions are initiated by the addition of isoprenyl-protein transferase and stopped at timed intervals (typically 15 min) by the addition of 1 M HCl in ethanol (1 mL). The quenched reactions are allowed to stand for 15 m (to complete the precipitation process). After adding 2 mL of 100% ethanol, the reactions are vacuum-filtered through Whatman GF/C filters. Filters are washed four times with 2 mL aliquots of 100% ethanol, mixed with scintillation fluid (10 mL) and then counted in a Beckman LS3801 scintillation counter.

For inhibition studies, assays are run as described above, except inhibitors are prepared as concentrated solutions in 100% dimethyl sulfoxide and then diluted 20-fold into the enzyme assay mixture. Substrate concentrations for inhibitor IC₅₀ determinations are as follows: FTase, 650 nM Ras-CVLS (SEQ.ID.NO.: 1), 100 nM farnesyl diphosphate.

The compounds of the instant invention described in the above Examples 1-2 were tested for inhibitory activity against human FPTase by the assay described above and were found to have an IC₅₀ of ≦5 μM.

### Example 4

Modified In vitro GGTase inhibition assay

The modified geranylgeranyl-protein transferase inhibition assay is carried out at room temperature. A typical reaction contains (in a final volume of 50 μL): [³H]geranylgeranyl diphosphate, biotinylated Ras peptide, 50 mM HEPES, pH 7.5, a modulating anion (for example 10 mM glycerophosphate or SmM ATP), 5 mM MgCL₂, 10 μM ZnCl₂, 0.1% PEG (15,000-20,000 mw), 2 mM dithiothreitol, and geranylgeranyl-protein transferase type I(GGTase). The GGTase-type I enzyme employed in the assay is prepared as described in U.S. Pat. No. 5,470,832, incorporated by reference. The Ras peptide is derived from the K4B-Ras protein and has the following sequence: biotinyl-GKKKKKKSKTKCVIM (single amino acid code) (SEQ.ID.NO.: 2). Reactions are initiated by the addition of GGTase and stopped at timed intervals (typically 15 min) by the addition of 200 μL of a 3 mg/mL suspension of streptavidin SPA beads (Scintillation Proximity Assay beads, Amersham) in 0.2 M sodium phosphate, pH 4, containing 50 mM EDTA, and 0.5% BSA. The quenched reactions are allowed to stand for 2 hours before analysis on a Packard TopCount scintillation counter.

For inhibition studies, assays are run as described above, except inhibitors are prepared as concentrated solutions in 100% dimethyl sulfoxide and then diluted 25-fold into the enzyme assay mixture. IC₅₀ values are determined with Ras peptide near KM concentrations. Enzyme and substrate concentrations for inhibitor IC₅₀ determinations are as follows: 75 pM GGTase-I, 1.6 μM Ras peptide, 100 nM geranylgeranyl diphosphate.

The compounds of the instant invention are tested for inhibitory activity against human GGTase type I by the assay described above.

### Example 5

Cell-based In Vitro Ras Farnesylation Assay

The cell line used in this assay is a v-ras line derived from either Ratl or NIH3T3 cells, which expressed viral Ha-ras p21. The assay is performed essentially as described in DeClue, J. E. et al., Cancer Research 51:712-717, (1991). Cells in 10 cm dishes at 50-75% confluency are treated with the test compound (final concentration of solvent, methanol or dimethyl sulfoxide, is 0.1%). After 4 hours at 37° C., the cells are labeled in 3 ml methionine-free DMEM supple-mented with 10% regular DMEM, 2% fetal bovine serum and 400 μCi[³⁵S]methionine (1000 Ci/mmol). After an additional 20 hours, the cells are lysed in 1 ml lysis buffer (1% NP40/20 mM HEPES, pH 7.5/5 mM MgCl₂/1 mM DTT/10 mg/ml aprotinen/2 mg/ml leupeptin/2 mg/ml antipain/0.5 mM PMSF) and the lysates cleared by centrifugation at 100,000×g for 45 min. Aliquots of lysates containing equal numbers of acid-precipitable counts are bought to 1 ml with IP buffer (lysis buffer lacking DTT) and immunoprecipitated with the ras-specific monoclonal antibody Y13-259 (Furth, M. E. et al., J. Virol. 43:294-304, (1982)). Following a 2 hour antibody incubation at 4° C., 200 μl of a 25% suspension of protein A-Sepharose coated with rabbit anti rat IgG is added for 45 min. The immunoprecipitates are washed four times with IP buffer (20 nM HEPES, pH 7.5/1 mM EDTA/1% Triton X-100.0.5% deoxycholate/0.1%/SDS/0.1 M NaCl) boiled in SDS-PAGE sample buffer and loaded on 13% acrylamide gels. When the dye front reached the bottom, the gel is fixed, soaked in Enlightening, dried and autoradiographed. The intensities of the bands corresponding to farnesylated and nonfarnesylated ras proteins are compared to determine the percent inhibition of farnesyl transfer to protein.

### Example 6

Cell-based In Vvitro Growth Inhibition Assay

To determine the biological consequences of FPTase inhibition, the effect of the compounds of the instant invention on the anchorage-independent growth of Ratl cells transformed with either a v-ras, v-raf, or v-mos oncogene is tested. Cells transformed by v-Raf and v-Mos maybe included in the analysis to evaluate the specificity of instant compounds for Ras-induced cell transformation.

Rat 1 cells transformed with either v-ras, v-raf, or v-mos are seeded at a density of 1×10⁴ cells per plate (35 mm in diameter) in a 0.3% top agarose layer in medium A (Dulbecco's modified Eagle's medium supplemented with 10% fetal bovine serum) over a bottom agarose layer (0.6%). Both layers contain 0.1% methanol or an appropriate concentration of the instant compound (dissolved in methanol at 1000 times the final concentration used in the assay). The cells are fed twice weekly with 0.5 ml of medium A containing 0.1% methanol or the concentration of the instant compound. Photomicrographs are taken 16 days after the cultures are seeded and comparisons are made.

### Example 7

Construction of SEAP Reporter Plasmid pDSE100

The SEAP reporter plasmid, pDSE100 was constructed by ligating a restriction fragment containing the SEAP coding sequence into the plasmid pCMV-RE-AKI. The SEAP gene is derived from the plasmid pSEAP2-Basic (Clontech, Palo Alto, Calif.). The plasmid pCMV-RE-AKI was constructed by Deborah Jones (Merck) and contains 5 sequential copies of the ‘dyad symmetry response element’ cloned upstream of a ‘CAT-TATA’ sequence derived from the cytomegalovirus immediate early promoter. The plasmid also contains a bovine growth hormone poly-A sequence.

The plasmid, pDSE100 was constructed as follows. A restriction fragment encoding the SEAP coding sequence was cut out of the plasmid pSEAP2-Basic using the restriction enzymes EcoRl and Hpal. The ends of the linear DNA fragments were filled in with the Klenow fragment of E. coli DNA Polymerase I. The ‘blunt ended’ DNA containing the SEAP gene was isolated by electrophoresing the digest in an agarose gel and cutting out the 1694 base pair fragment. The vector plasmid pCMV-RE-AKI was linearized with the restriction enzyme Bgl-II and the ends filled in with Klenow DNA Polymerase I. The SEAP DNA fragment was blunt end ligated into the pCMV-RE-AKI vector and the ligation products were transformed into DH5-alpha E. coli cells (Gibco-BRL). Transformants were screened for the proper insert and then mapped for restriction fragment orientation. Properly oriented recombinant constructs were sequenced across the cloning junctions to verify the correct sequence. The resulting plasmid contains the SEAP coding sequence downstream of the DSE and CAT-TATA promoter elements and upstream of the BGH poly-A sequence.

Alternative Construction of SEAP Reporter Plasmid, pDSE101

The SEAP repotrer plasmid, pDSE101 is also constructed by ligating a restriction fragment containing the SEAP coding sequence into the plasmid pCMV-RE-AKI. The SEAP gene is derived from plasmid pGEM7zf−)/SEAP.

The plasmid pDSE101 was constructed as follows: A restriction fragment containing part of the SEAP gene coding sequence was cut out of the plasmid pGEM7zf(−)/SEAP using the restriction enzymes Apa I and KpnI. The ends of the linear DNA fragments were chewed back with the Klenow fragment of E. coli DNA Polymerase I. The “blunt ended” DNA containing the truncated SEAP gene was isolated by electrophoresing the digest in an agarose gel and cutting out the 1910 base pair fragment. This 1910 base pair fragment was ligated into the plasmid pCMV-RE-AKI which had been cut with Bgl-ll and filled in with E. coli Klenow fragment DNA polymerase. Recombinant plasmids were screened for insert orientation and sequenced through the ligated junctions. The plasmid pCMV-RE-AKI is derived from plasmid pCMVIE-AKI-DBFR (Whang, Y., Silberklang, M., Morgan, A., Munshi, S., Lenny, A. B., Ellis, R. W., and Kieff, E. (1987) J. Virol., 61, 1796-1807) by removing an EcoRI fragment containing the DHFR and Neomycin markers. Five copies of the fos promoter serum response element were inserted as described previously (Jones, R. E., Defeo-Jones, D., McAvoy, E. M., Vuocolo, G. A., Wegrzyn, R. J., Haskell, K. M. and Oliff, A. (1991) Oncogene, 6, 745-751) to create plasmid pCMV-RE-AKI. The plasmid pGEM7zf(−)/SEAP was constructed as follows. The SEAP gene was PCRed, in two segments from a human placenta cDNA library (Clontech) using the following oligos.

Sense strand N-terminal SEAP: 5′ GAGAGGGAATTCGGGCCCTTCCTGCAT GCTGCTGCTGCTGCTGCTGCTGGGC 3′ (SEQ.ID.NO.:3)

Antisense strand N-terminal SEAP: 5′ GAGAGAGCTCGAGGTTAACCCGGGT GCGCGGCGTCGGTGGT 3′ (SEQ.ID.NO.:4)

Sense strand C-terminal SEAP: 5′ GAGAGAGTCTAGAGTTAACCCGTGGTCC CCGCGTTGCTTCCT 3′ (SEQ.ID.NO.:5)

Antisense strand C-terminal SEAP: 5′ GAAGAGGAAGCTTGGTACCGCCACTG GGCTGTAGGTGGTGGCT 3′ (SEQ.ID.NO.:6)

The N-terminal oligos (SEQ.ID.NO.: 4 and SEQ.ID.NO.: 5) were used to generate a 1560 bp N-terminal PCR product that contained EcoRI and HpaI restriction sites at the ends. The Antisense N-terminal oligo (SEQ.ID.NO.: 4) introduces an internal translation STOP codon within the SEAP gene along with the Hpal site. The C-terminal oligos (SEQ.ID.NO.: 5 and SEQ.ID.NO.: 6) were used to amplify a 412 bp C-terminal PCR product containing HpaI and HindIII restriction sites. The sense strand C-terminal oligo (SEQ.ID.NO.: 5) introduces the internal STOP codon as well as the Hpal site. Next, the N-terminal amplicon was digested with EcoRI and Hpal while the C-terminal amplicon was digested with Hpal and HindIII. The two fragments comprising each end of the SEAP gene were isolated by electrophoresing the digest in an agarose gel and isolating the 1560 and 412 base pair fragments. These two fragments were then co-ligated into the vector pGEM7zf(−) (Promega) which had been restriction digested with EcoRI and HindIII and isolated on an agarose gel. The resulting clone, pGEM7zf(−)/SEAP contains the coding sequence for the SEAP gene from amino acids.

Construction of a Constitutively expressing SEAP plasmid pCMV-SEAP

An expression plasmid constitutively expressing the SEAP protein was created by placing the sequence encoding a truncated SEAP gene downstream of the cytomegalovirus (CMV) IE-1 promoter. The expression plasmid also includes the CMV intron A region 5′ to the SEAP gene as well as the 3′ untranslated region of the bovine growth hormone gene 3′ to the SEAP gene.

The plasmid pCMVIE-AKI-DHFR (Whang et al, 1987) containing the CMV immediate early promoter was cut with EcoRI generating two fragments. The vector fragment was isolated by agarose electrophoresis and religated. The resulting plasmid is named pCMV-AKI. Next, the cytomegalovirus intron A nucleotide sequence was inserted downstream of the CMV IE1 promter in pCMV-AKI. The intron A sequence was isolated from a genomic clone bank and subcloned into pBR322 to generate plasmid pl6T-286. The intron A sequence was mutated at nucleotide 1856 (nucleotide numbering as in Chapman, B.S., Thayer, R. M., Vincent, K. A. and Haigwood, N. L., Nuc.Acids Res. 19, 3979-3986) to remove a SacI restriction site using site directed mutagenesis. The mutated intron A sequence was PCRed from the plasmid pl6T-287 using the following oligos.

Sense strand: 5′ GGCAGAGCTCGTTTAGTGAACCGTCAG 3′ (SEQ.ID.NO.: 7)

Antisense strand: 5′ GAGAGATCTCAAGGACGGTGACTGCAG 3′ (SEQ.ID.NO.: 8)

These two oligos generate a 991 base pair fragment with a SacI site incorporated by the sense oligo and a Bgl-II fragment incorporated by the antisense oligo. The PCR fragment is trimmed with SacI and Bgl-II and isolated on an agarose gel. The vector pCMV-AKI is cut with SacI and Bgl-II and the larger vector fragment isolated by agarose gel electrophoresis. The two gel isolated fragments are ligated at their respective SacI and Bgl-II sites to create plasmid pCMV-AKI-InA. The DNA sequence encoding the truncated SEAP gene is inserted into the pCMV-AKI-InA plasmid at the Bgl-II site of the vector. The SEAP gene is cut out of plasmid pGEM7zf(−)/SEAP (described above) using EcoRI and HindIII. The fragment is filled in with Klenow DNA polymerase and the 1970 base pair fragment isolated from the vector fragment by agarose gel electrophoresis. The pCMV-AKI-InA vector is prepared by digesting with Bgl-II and filling in the ends with Klenow DNA polymerase. The final construct is generated by blunt end ligating the SEAP fragment into the pCMV-AKI-InA vector. Transformants were screened for the proper insert and then mapped for restriction fragment orientation. Properly oriented recombinant constructs were sequenced across the cloning junctions to verify the correct sequence. The resulting plasmid, named pCMV-SEAP, contains a modified SEAP sequence downstream of the cytomegalovirus immediately early promoter IE-1 and intron A sequence and upstream of the bovine growth hormone poly-A sequence. The plasmid expresses SEAP in a constitutive manner when transfected into mammalian cells.

Cloning of a Myristylated viral-H-ras Expression Plasmid

A DNA fragment containing viral-H-ras can be PCRed from plasmid “H-1” (Ellis R. et al. J. Virol. 36, 408, 1980) or “HB-11 (deposited in the ATCC under Budapest Treaty on Aug. 27, 1997, and designated ATCC 209,218) using the following oligos.

Sense strand:

5′TCTCCTCGAGGCCACCATGGGGAGTAGCAAGAGCAAGCCTAAGGACCC CAGCCAGCGCCGGATGACAGAATACAAGCTTGTGGTGG 3′. (SEQ.ID.NO.: 9)

Antisense:

5′CACATCTAGATCAGGACAGCACAGACTTGCAGC 3′. (SEQ.ID.NO.: 10)

A sequence encoding the first 15 aminoacids of the v-src gene, containing a myristylation site, is incorporated into the sense strand oligo. The sense strand oligo also optimizes the ‘Kozak’ translation initiation sequence immediately 5′ to the ATG start site. To prevent prenylation at the viral-ras C-terminus, cysteine 186 would be mutated to a serine by substituting a G residue for a C residue in the C-terminal antisense oligo. The PCR primer oligos introduce an XhoI site at the 5′ end and a Xbai site at the 3′end. The XhoI-Xbal fragment can be ligated into the mammalian expression plasmid pCI (Promega) cut with XhoI and XbaI. This results in a plasmid in which the recombinant myr-viral-H-ras gene is constitutively transcribed from the CMV promoter of the pCI vector.

Cloning of a viral-H-ras-CVLL Expression Plasmid

A viral-H-ras clone with a C-terminal sequence encoding the amino acids CVLL can be cloned from the plasmid “H-1” (Ellis R., et al. J. Virol. 36, 408, 1980) or “HB-11 (deposited in the ATCC under Budapest Treaty on Aug. 27, 1997, and designated ATCC 209,218) by PCR using the following oligos.

Sense strand:

5′ TCTCCTCGAGGCCACCATGACAGAATACAAGCTTGTGGTGG-3′ (SEQ.ID.NO.: 11)

Antisense strand:

5′CACTCTAGACTGGTGTCAGAGCAGCACACACTTGCAGC-3′ (SEQ.ID.NO.: 12)

The sense strand oligo optimizes the ‘Kozak’ sequence and adds an XhoI site. The antisense strand mutates serine 189 to leucine and adds an Xbal site. The PCR fragment can be trimmed with XhoI and Xbal and ligated into the XhoI-Xbal cut vector pCI (Promega). This results in a plasmid in which the mutated viral-H-ras-CVLL gene is constitutively transcribed from the CMV promoter of the pCI vector.

Cloning of c-H-ras-Leu61 Expression Plasmid

The human c-H-ras gene can be PCRed from a human cerebral cortex cDNA library (Clontech) using the following oligonucleotide primers.

Sense strand:

5′-GAGAGAATTCGCCACCATGACGGAATATAAGCTGGTGG-3′ (SEQ.ID.NO.: 13)

Antisense strand:

5′-GAGAGTCGACGCGTCAGGAGAGCACACACTTGC-3′ (SEQ.ID.NO.: 14)

The primers will amplify a c-H-ras encoding DNA fragment with the primers contributing an optimized ‘Kozak’ translation start sequence, an EcoRI site at the N-terminus and a Sal I stite at the C-terminal end. After trimming the ends of the PCR product with EcoRI and Sal I, the c-H-ras fragment can be ligated ligated into an EcoRI -Sal I cut mutagenesis vector pAlter-1 (Promega). Mutation of glutamine-61 to a leucine can be accomplished using the manufacturer's protocols and the following oligonucleotide:

5′-CCGCCGGCCTGGAGGAGTACAG-3′ (SEQ.ID.NO.: 15)

After selection and sequencing for the correct nucleotide substitution, the mutated c-H-ras-Leu61 can be excised from the pAlter-1 vector, using EcoRI and Sal I, and be directly ligated into the vector pCI (Promega) which has been digested with EcoRI and Sal I. The new recombinant plasmid will constitutively transcribe c-H-ras-Leu61 from the CMV promoter of the pCI vector.

Cloning of a c-N-ras-Val-12 Expression Plasmid

The human c-N-ras gene can be PCRed from a human cerebral cortex cDNA library (Clontech) using the following oligonucleotide primers.

Sense strand:

5′ -GAGAGAATTCGCCACCATGACTGAGTACAAACTGGTGG-3′ (SEQ.ID.NO.: 16)

Antisense strand:

5′-GAGAGTCGACTTGTTACATCACCACACATGGC-3′ (SEQ.ID.NO.: 17)

The primers will amplify a c-N-ras encoding DNA fragment with the primers contributing an optimized ‘Kozak’ translation start sequence, an EcoRI site at the N-terminus and a Sal I stite at the C-terminal end. After trimming the ends of the PCR product with EcoRI and Sal I, the c-N-ras fragment can be ligated into an EcoRI -Sal I cut mutagenesis vector pAlter-1 (Promega). Mutation of glycine-12 to a valine can be accomplished using the manufacturer's protocols and the following oligonucleotide:

5′-GTTGGAGCAGTTGGTGTTGGG-3′ (SEQ.ID.NO.: 18)

After selection and sequencing for the correct nucleotide substitution, the mutated c-N-ras-Val-12 can be excised from the pAlter-1 vector, using EcoRI and Sal I, and be directly ligated into the vector pCI (Promega) which has been digested with EcoRI and Sal I. The new recombinant plasmid will constitutively transcribe c-N-ras-Val-12 from the CMV promoter of the pCI vector.

Cloning of a c-K-ras-Val-12 Expression Plasmid

The human c-K-ras gene can be PCRed from a human cerebral cortex cDNA library (Clontech) using the following oligonucleotide primers.

Sense strand:

5′-GAGAGGTACCGCCACCATGACTGAATATAAACTTGTGG-3′ (SEQ.ID.NO.: 19)

Antisense strand:

5′ -CTCTGTCGACGTATTTACATAATTACACACTTTGTC-3′ (SEQ.ID .NO.: 20)

The primers will amplify a c-K-ras encoding DNA fragment with the primers contributing an optimized ‘Kozak’ translation start sequence, a KpnI site at the N-terminus and a Sal I stite at the C-terminal end. After trimming the ends of the PCR product with Kpn I and Sal I, the c-K-ras fragment can be ligated into a KpnI -Sal I cut mutagenesis vector pAlter-1 (Promega). Mutation of cysteine-12 to a valine can be accomplished using the manufacturer's protocols and the following oligonucleotide:

5′-GTAGTTGGAGCTGTTGGCGTAGGC-3′ (SEQ.ID .NO.: 21)

After selection and sequencing for the correct nucleotide substitution, the mutated c-K-ras-Val-12 can be excised from the pAlter-1 vector, using KpnI and Sal I, and be directly ligated into the vector pCI (Promega) which has been digested with KpnI and Sal I. The new recombinant plasmid will constitutively transcribe c-K-ras-Val-12 from the CMV promoter of the pCI vector.

SEAP Assay

Human C₃₃A cells (human epitheial carcenoma—ATTC collection) are seeded in 10 cm tissue culture plates in DMEM+10% fetal calf serum +1× Pen/Strep+1× glutamine+1× NEAA. Cells are grown at 37° C. in a 5% CO₂ atmosphere until they reach 50-80% of confluency.

The transient transfection is performed by the CaPO4 method (Sambrook et al., 1989). Thus, expression plasmids for H-ras, N-ras, K-ras, Myr-ras or H-ras-CVLL are co-precipitated with the DSE-SEAP reporter construct. For 10 cm plates 600 μl of CaCl₂-DNA solution is added dropwise while vortexing to 600 μl of 2× HBS buffer to give 1.2 ml of precipitate solution (see recipes below). This is allowed to sit at room temperature for 20 to 30 minutes. While the precipitate is forming, the media on the C33A cells is replaced with DMEM (minus phenol red; Gibco cat. #31053-028)+0.5% charcoal stripped calf serum +1× (Pen/Strep, Glutamine and nonessential aminoacids). The CaPO₄-DNA precipitate is added dropwise to the cells and the plate rocked gently to distribute. DNA uptake is allowed to proceed for 5-6 hrs at 37° C. under a 5% CO₂ atmosphere.

Following the DNA incubation period, the cells are washed with PBS and trypsinized with lml of 0.05% trypsin. The 1 ml of trypsinized cells is diluted into lOml of phenol red free DMEM+0.2% charcoal stripped calf serum +1× (Pen/Strep, Glutamine and NEAA). Transfected cells are plated in a 96 well micro-titer plate (100 μl/well) to which drug, diluted in media, has already been added in a volume of 100 μl. The final volume per well is 200 μl with each drug concentration repeated in triplicate over a range of half-log steps.

Incubation of cells and drugs is for 36 hrs at 37° C. under C₀₂. At the end of the incubation period, cells are examined microscopically for evidence of cell distress. Next, 100 μl of media containing the secreted alkaline phosphatase is removed from each well and transferred to a microtube array for heat treatment at 65° C. for 1 hr to inactivate endogenous alkaline phosphatases (but not the heat stable secreted phosphatase).

The heat treated media is assayed for alkaline phosphatase by a luminescence assay using the luminescence reagent CSPD® (Tropix, Bedford, Mass.). A volume of 50 μl media is combined with 200 μl of CSPD cocktail and incubated for 60 minutes at room temperature. Luminesence is monitored using an ML2200 microplate luminometer (Dynatech). Luminescence reflects the level of activation of the fos reporter construct stimulated by the transiently expressed protein.

DNA-CaPO₄ Precipitate for 10 cm. Plate of Cells

| | | |
|---|---|---|
| Ras expression plasmid (1 μg/μl) | 10 | μl |
| DSE-SEAP Plasmid (1 μg/μl) | 2 | μl |
| Sheared Calf Thymus DNA (1 μg/μl) | 8 | μl |
| 2M CaCl₂ | 74 | μl |
| dH₂O | 506 | μl |

2× HBS Buffer

280 mM NaCl

10 mM KCl

1.5 mM Na₂HPO₄ 2H₂O

12 mM dextrose

50 mM HEPES

Final pH=7.05

Luminesence Buffer (26 ml)

| | | |
|---|---|---|
| Assay Buffer | 20 | ml |
| Emerald Reagent ™ (Tropix) | 2.5 | ml |
| 100 mM homoarginine | 2.5 | ml |
| CSPD Reagent ® (Tropix) | 1.0 | ml |

Assay Buffer

Add 0.05M Na₂CO₃ to 0.05M NaHCO₃ to obtain pH 9.5.

Make 1 mM in MgCl₂

### Example 8

The processing assays employed are modifications of that described by DeClue et al [Cancer Research 51, 712-717, 1991].

K4B-Ras Processing Inhibition Assay

PSN-1 (human pancreatic carcinoma) or viral-K4B-ras-transformed Ratl cells are used for analysis of protein processing. Subconfluent cells in 100 mm dishes are fed with 3.5 ml of media (methionine-free RPMI supplemented with 2% fetal bovine serum or cysteine-free/methionine-free DMEM supplemented with 0.035 ml of 200 mM glutamine (Gibco), 2% fetal bovine serum, respectively) containing the desired concentration of test compound, lovastatin or solvent alone. Cells treated with lovastatin (5-10 μM), a compound that blocks Ras processing in cells by inhibiting a rate-limiting step in the isoprenoid biosynthetic pathway, serve as a positive control. Test compounds are prepared as 1000× concentrated solutions in DMSO to yield a final solvent concentration of 0.1%. Following incubation at 37° C. for two hours 204 μCi/ml [³⁵S]Pro-Mix (Amersham, cell labeling grade) is added.

After introducing the label amino acid mixture, the cells are incubated at 37° C. for an additional period of time (typically 6 to 24 hours). The media is then removed and the cells are washed once with cold PBS. The cells are scraped into 1 ml of cold PBS, collected by centrifugation (10,000×g for 10 sec at room temperature), and lysed by vortexing in 1 ml of lysis buffer (1% Nonidet P-40, 20 mM HEPES, pH 7.5, 150 mM NaCl, 1 mM EDTA, 0.5% deoxycholate, 0.1% SDS, 1 mM DTT, 10 μg/ml AEBSF, 10 μg/ml aprotinin, 2 μg/ml leupeptin and 2 μg/ml antipain). The lysate is then centrifuged at 15,000×g for 10 min at 4° C. and the supernatant saved.

For immunoprecipitation of Ki4B-Ras, samples of lysate supernatant containing equal amounts of protein are utilized. Protein concentration is determined by the bradford method utilizing bovine serum albumin as a standard. The appropriate volume of lysate is brought to 1 ml with lysis buffer lacking DTT and 8 jig of the pan Ras monoclonal antibody, Y13-259, added. The protein/antibody mixture is incubated on ice at 4° C. for 24 hours. The immune complex is collected on pansorbin (Calbiochem) coated with rabbit antiserum to rat IgG (Cappel) by tumbling at 4° C. for 45 minutes. The pellet is washed 3 times with 1 ml of lysis buffer lacking DTT and protease inhibitors and resuspended in 100 μl elution buffer (10 mM Tris pH 7.4, 1% SDS). The Ras is eluted from the beads by heating at 95° C. for 5 minutes, after which the beads are pelleted by brief centrifugation (15,000×g for 30 sec. at room temperature).

The supernatant is added to 1 ml of Dilution Buffer 0.1% Triton X-100, 5 mM EDTA, 50 mM NaCl, 10 mM Tris pH 7.4) with 2 μg Kirsten-ras specific monoclonal antibody, c-K-ras Ab-1 (Calbiochem). The second protein/antibody mixture is incubated on ice at 4° C. for 1-2 hours. The immune complex is collected on pansorbin (Calbiochem) coated with rabbit antiserum to rat IgG (Cappel) by tumbling at 4° C. for 45 minutes. The pellet is washed 3 times with 1 ml of lysis buffer lacking DTT and protease inhibitors and resuspended in Laemmli sample buffer. The Ras is eluted from the beads by heating at 95° C. for 5 minutes, after which the beads are pelleted by brief centrifugation. The supernatant is subjected to SDS-PAGE on a 12% acrylamide gel (bis-acrylamide:acrylamide, 1: 100), and the Ras visualized by fluorography.

hDJ Processing Inhibition Assay

PSN-1 cells are seeded in 24-well assay plates. For each compound to be tested, the cells are treated with a minimum of seven concentrations in half-log steps. The final solvent (DMSO) concentration is 0.1%. A vehicle-only control is included on each assay plate. The cells are treated for 24 hours at 37° C./5% CO₂.

The growth media is then aspirated and the samples are washed with PBS. The cells are lysed with SDS-PAGE sample buffer containing 5% 2-mercaptoethanol and heated to 95° C. for 5 minutes. After cooling on ice for 10 minutes, a mixture of nucleases is added to reduce viscosity of the samples.

The plates are incubated on ice for another 10 minutes. The samples are loaded onto pre-cast 8% acrylamide gels and electrophoresed at 15 mA/gel for 3-4 hours. The samples are then transferred from the gels to PVDF membranes by Western blotting.

The membranes are blocked for at least 1 hour in buffer containing 2% nonfat dry milk. The membranes are then treated with a monoclonal antibody to hDJ-2 (Neomarkers Cat. # MS-225), washed, and treated with an alkaline phosphatase-conjugated secondary antibody. The membranes are then treated with a fluorescent detection reagent and scanned on a phosphorimager.

For each sample, the percent of total signal corresponding to the unprenylated species of hDJ (the slower-migrating species) is calculated by densitometry. Dose-response curves and EC₅₀ values are generated using 4-parameter curve fits in SigmaPlot software.

### Example 9

Rap1 Processing Inhibition Assay

Protocol A:

Cells are labeled, incubated and lysed as described in Example 8.

For immunoprecipitation of Rap1, samples of lysate supernatant containing equal amounts of protein are utilized. Protein concentration is determined by the bradford method utilizing bovine serum albumin as a standard. The appropriate volume of lysate is brought to 1 ml with lysis buffer lacking DTT and 2 μg of the Rap1 antibody, Rap1/Krev1 (121) (Santa Cruz Biotech), is added. The protein/antibody mixture is incubated on ice at 4° C. for 1 hour. The immune complex is collected on pansorbin (Calbiochem) by tumbling at 4° C. for 45 minutes. The pellet is washed 3 times with 1 ml of lysis buffer lacking DTT and protease inhibitors and resuspended in 100 μl elution buffer (10 mM Tris pH 7.4, 1% SDS). The Rap1 is eluted from the beads by heating at 95° C. for 5 minutes, after which the beads are pelleted by brief centrifugation (15,000×g for 30 sec. at room temperature).

The supernatant is added to 1 ml of Dilution Buffer (0.1% Triton X-100, 5 mM EDTA, 50 mM NaCl, 10 mM Tris pH 7.4) with 2 μg Rap1 antibody, Rap1/Krev1 (121) (Santa Cruz Biotech). The second protein/antibody mixture is incubated on ice at 4° C. for 1-2 hours. The immune complex is collected on pansorbin (Calbiochem) by tumbling at 4° C. for 45 minutes. The pellet is washed 3 times with 1 ml of lysis buffer lacking DTT and protease inhibitors and resuspended in Laemmli sample buffer. The Rap1 is eluted from the beads by heating at 95° C. for 5 minutes, after which the beads are pelleted by brief centrifugation. The supernatant is subjected to SDS-PAGE on a 12% acrylamide gel (bis-acrylamide:acrylamide, 1:100), and the Rap1 visualized by fluorography.

Protocol B:

PSN-1 cells are passaged every 3-4 days in 10 cm plates, splitting near-confluent plates 1:20 and 1:40. The day before the assay is set up, 5×10⁶ cells are plated on 15 cm plates to ensure the same stage of confluency in each assay. The media for these cells is RPM1 1640 (Gibco), with 15% fetal bovine serum and 1× Pen/Strep antibiotic mix.

The day of the assay, cells are collected from the 15 cm plates by trypsinization and diluted to 400,000 cells/ml in media. 0.5 ml of these diluted cells are added to each well of 24-well plates, for a final cell number of 200,000 per well. The cells are then grown at 37° C. overnight.

The compounds to be assayed are diluted in DMSO in ½-log dilutions. The range of final concentrations to be assayed is generally 0.1-100 μM. Four concentrations per compound is typical. The compounds are diluted so that each concentration is 1000× of the final concentration (i.e., for a 10 μM data point, a 10 mM stock of the compound is needed).

2 μL of each 1000× compound stock is diluted into lml media to produce a 2× stock of compound. A vehicle control solution (2 μL DMSO to lml media), is utilized. 0.5 ml of the 2× stocks of compound are added to the cells.

After 24 hours, the media is aspirated from the assay plates. Each well is rinsed with Iml PBS, and the PBS is aspirated. 180 μL SDS-PAGE sample buffer (Novex) containing 5% 2-mercaptoethanol is added to each well. The plates are heated to 100° C. for 5 minutes using a heat block containing an adapter for assay plates. The plates are placed on ice. After 10 minutes, 20 μL of an RNAse/DNase mix is added per well. This mix is 1 mg/ml DNaseI (Worthington Enzymes), 0.25 mg/ml Rnase A (Worthington Enzymes), 0.5M Tris-HCl pH8.0 and 50 mM MgCl₂. The plate is left on ice for 10 minutes. Samples are then either loaded on the gel, or stored at −70° C. until use.

Each assay plate (usually 3 compounds, each in 4-point titrations, plus controls) requires one 15-well 14% Novex gel. 25 μl of each sample is loaded onto the gel. The gel is run at 15 mA for about 3.5 hours. It is important to run the gel far enough so that there will be adequate separation between 21 kd (Rap1) and 29 kd (Rab6).

The gels are then transferred to Novex pre-cut PVDF membranes for 1.5 hours at 30V (constant voltage). Immediately after transferring, the membranes are blocked overnight in 20 ml Western blocking buffer (2% nonfat dry milk in Western wash buffer (PBS +0.1% Tween-20). If blocked over the weekend, 0.02% sodium azide is added. The membranes are blocked at 4° C. with slow rocking.

The blocking solution is discarded and 20 ml fresh blocking solution containing the anti Rap1a antibody (Santa Cruz Biochemical SC₁₄₈₂) at 1:1000 (diluted in Western blocking buffer) and the anti Rab6 antibody (Santa Cruz Biochemical SC₃₁₀) at 1:5000 (diluted in Western blocking buffer) are added. The membranes are incubated at room temperature for 1 hour with mild rocking. The blocking solution is then discarded and the membrane is washed 3 times with Western wash buffer for 15 minutes per wash. 20 ml blocking solution containing 1:1000 (diluted in Western blocking buffer) each of two alkaline phosphatase conjugated antibodies (Alkaline phosphatase conjugated Anti-goat IgG and Alkaline phosphatase conjugated anti-rabbit IgG [Santa Cruz Biochemical]) is then added. The membrane is incubated for one hour and washed 3x as above.

About 2 ml per gel of the Amersham ECF detection reagent is placed on an overhead transparency (ECF) and the PVDF membranes are placed face down onto the detection reagent. This is incubated for one minute, then the membrane is placed onto a fresh transparency sheet.

The developed transparency sheet is scanned on a phosphorimager and the Rap1a Minimum Inhibitory Concentration is determined from the lowest concentration of compound that produces a detectable Rap1a Western signal. The Rap1a antibody used recognizes only unprenylated/unprocessed Rap1a, so that the precence of a detectable Rap1a Western signal is indicative of inhibition of Rap1a prenylation.

Protocol C

This protocol allows the determination of an EC₅₀ for inhibition of processing of Rap1a. The assay is run as described in Protocol B with the following modifications. 20 μl of sample is run on pre-cast 10-20% gradient acrylamide mini gels (Novex Inc.) at 15 mA/gel for 2.5-3 hours. Prenylated and unprenylated forms of Rap1a are detected by blotting with a polyclonal antibody (Rap1/Krev-1 Ab#121; Santa Cruz Research Products #sc-65), followed by an alkaline phosphatase-conjugated anti-rabbit IgG antibody. The percentage of unprenylated Rap1a relative to the total amount of Rap1a is determined by peak integration using Imagequant® software (Molecular Dynamics). Unprenylated Rap1a is distinguished from prenylated protein by virtue of the greater apparent molecular weight of the prenylated protein. Dose-response curves and EC₅₀ values are generated using 4-parameter curve fits in SigmaPlot software.

### Example 10

In Vivo Tumor Growth Inhibition Assay (Nude Mouse)

In vivo efficacy as an inhibitor of the growth of cancer cells may be confirmed by several protocols well known in the art. Examples of such in vivo efficacy studies are described by N. E. Kohl et al. (Nature Medicine, 1:792-797 (1995)) and N. E. Kohl et al. (Proc. Nat. Acad. Sci. U.S.A., 91:9141-9145 (1994)).

Rodent fibroblasts transformed with oncogenically mutated human Ha-ras or Ki-ras (10⁶ cells/animal in 1 ml of DMEM salts) are injected subcutaneously into the left flank of 8-12 week old female nude mice (Harlan) on day 0. The mice in each oncogene group are randomly assigned to a vehicle, compound or combination treatment group. Animals are dosed subcutaneously starting on day 1 and daily for the duration of the experiment. Alternatively, the farnesyl-protein transferase inhibitor may be administered by a continuous infusion pump. Compound, compound combination or vehicle is delivered in a total volume of 0.1 ml. Tumors are excised and weighed when all of the vehicle-treated animals exhibited lesions of 0.5-1.0 cm in diameter, typically 11-15 days after the cells were injected. The average weight of the tumors in each treatment group for each cell line is calculated.

What is claimed is:

1. A compound which is illustrated by formula A: 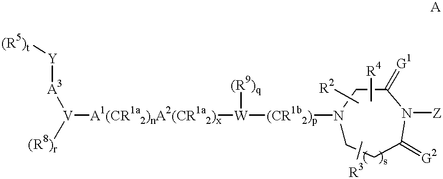
wherein:
R^{1a} and R^{1b} are independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, R¹⁰O—, R¹¹S(O)ₘ—, R¹⁰OC(O)NR¹⁰—, (R¹⁰)₂NC(O)—, (R¹⁰)₂NC(O)NR¹⁰—, CN, NO₂, R¹¹OC(O)—, R¹⁰OC(O)—, —N(R¹⁰)₂, or R¹¹OC(O)NR¹⁰—, or
c) unsubstituted or substituted C₁-C₆ alkyl wherein the sub stitutent on the substituted C₁-C₆ alkyl is selected from unsubstituted or substituted aryl, unsubstituted or substituted heterocycle, unsubstituted or substituted C₃-C₁₀ cycloalkyl, C₂-C₈ alkenyl, C₂-C₈ alkynyl, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, and R¹¹OC(O)NR¹⁰—;
R² and R³ are independently selected from: H, unsubstituted or substituted C₁₋₆ alkyl, unsubstituted or substituted C₂₋₈ alkenyl, unsubstituted or substituted C₂₋₈ alkynyl, unsubstituted or substituted aryl, unsubstituted or substituted heterocycle, 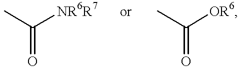
wherein the substituted group is substituted with one or more of:
1) aryl or heterocycle, unsubstituted or substituted with:
a) C₁₋₆ alkyl,
b) (CH₂)pOR⁶,
c) (CH₂)pNR⁶R⁷,
d) halogen,
e) CN,
2) C₃₋₆ cycloalkyl,
3) OR⁶,
4) SR^{6a}, S(O)R^{6a}, SO₂R^{6a},
5) —N R⁶R⁷ 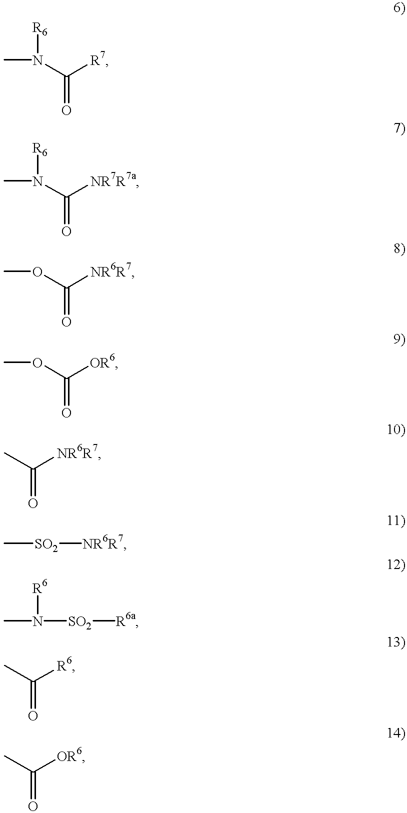
15) N₃, or
16) F; or
R² and R³ are attached to the same C atom and are combined to form -(CH₂)ᵤ— wherein one of the carbon atoms is optionally replaced by a moiety selected from: O, S(O)ₘ, —NC(O)—, and —N(COR¹⁰)—;
R⁴ is selected from H and unsubstituted or substituted C₁-C₆ alkyl;
and any two of R², R³ or R⁴ are optionally attached to the same carbon atom;
R⁵ is independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and
c) C₁-C₆ alkyl, unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;
R⁶, R⁷ and R^{7a} are independently selected from: H, C₁-C₆ alkyl, C₃₋₆ cycloalkyl, heterocycle, aryl, aroyl, heteroaroyl, arylsulfonyl, heteroarylsulfonyl, unsubstituted or substituted with:
a) C₁₋₆ alkoxy,
b) C₁-C₂₀ alkyl
c) aryl or heterocycle,
d) halogen,
e) HO,
f) —C(O)R¹¹,
g) —SO₂R¹¹, or
h) N(R¹⁰)₂; or
R⁶ and R⁷ may be joined in a ring;
R⁷ and R^{7a} may be joined in a ring;
R^{6a} is selected from: C₁-C₆ alkyl, C₃₋₆ cycloalkyl, heterocycle, aryl, aroyl, heteroaroyl, arylsulfonyl, heteroarylsulfonyl, unsubstituted or substituted with:
a) C₁₋₄ alkoxy,
b) C₁-C₂₀ alkyl
c) aryl or heterocycle,
d) halogen,
e) HO,
f) —C(O)R¹¹,
g) —SO₂R¹¹, or
h) N(R¹⁰)₂;
R⁸ is independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR10—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and
c) C₁-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;
R⁹ is selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, NO₂,
R¹⁰C(O)—, R¹⁰OC(O)—, —N(R¹⁰)₂, or R¹⁰C(O)NR¹⁰—, and
c) C₁-C₆ alkyl unsubstituted or substituted by aryl, heterocycle, C₃-C₁O cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹⁰C(O)NR¹⁰—;
R¹⁰ is independently selected from hydrogen, unsubstituted or substituted C₁-C₆ alkyl, perfluoroalkyl, unsubstituted or substituted aralkyl, and unsubstituted or substituted aryl;
R¹¹ is independently selected from unsubstituted or substituted C₁-C₆ alkyl and unsubstituted or substituted aryl;
A¹ and A² are independently selected from: a bond, —CH═CH—, —C≡C—, —C(O)—, —C(O)NR¹⁰, —NR¹⁰C(O)—, O, —N(R¹⁰), —S(O)₂N(R¹⁰), —N(R¹⁰)S(O)₂—, or S(O)ₘ;
A³ is selected from —C(O)—, —C(R^{1a})₂—, O, —N(R¹⁰)— and S(O)ₘ;
G¹ or G² is selected from H₂ or O, provided that if G¹ is O then G² is H₂ and if G² is O, then G¹ is H₂;
V is selected from:
a) heterocycle, and
b) aryl,
W is a heterocycle;
Y is heteroaryl;
Z is a unsubstituted or substituted group selected from aryl, heteroaryl, arylmethyl, heteroarylmethyl, arylsulfonyl, heteroarylsulfonyl, wherein the substituted group is substituted with one or more of the following:
1) C₁-C₆ alkyl, unsubstituted or substituted with:
a) C₁₋₆ alkoxy,
b) NR⁶R⁷,
c) C₃₋₆ cycloalkyl,
d) aryl or heterocycle,
e) HO,
f) —S(O)ₘR^{6a}, or
g) —C(O)NR⁶R⁷,
2) unsubstituted or substituted aryl or unsubstituted or substituted heterocycle,
3) halogen,
4) OR⁶,
5) NR⁶R⁷,
6) CN,
7) NO₂,
8) CF₃;
9) —S(O)ₘR^{6a},
10) —C(O)NR⁶R⁷,
11) —OCF₃,
12) unsubstituted or substituted C₁₋₆ alkoxy,
13) C₂-C₈ alkenyl,
14) C₂-C₈ alkynyl, or
15) C₃-C₁₀ cycloalkyl;
m is 0, 1 or 2;
n is 0, 1, 2, 3 or 4;
p is 0, 1, 2, 3 or 4;
q is 0, 1 or 2;
r is 0 to 5;
s is 0 or 1;
t is 0 to 5;
u is 4 or 5; and
x is 0, 1, 2, 3or 4;
or the pharmaceutically acceptable salts or optical isomers thereof.

2. The compound according to claim 1, as illustrated by formula B: 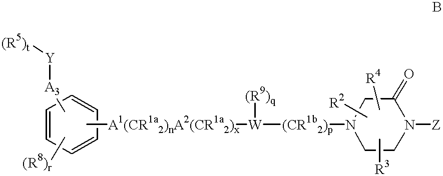
wherein:
R^{1a} and R^{1b} are independently selected from:
a) hydrogen,
b) unsubstituted or substituted aryl, unsubstituted or substituted heterocycle, unsubstituted or substituted C₃-C₁₀ cycloalkyl, R¹⁰O—, —N(R¹⁰)₂, or, C₂-C₈ alkenyl, or
c) unsubstituted or substituted C₁-C₆ alkyl wherein the substitutent on the substituted C₁-C₆ alkyl is selected from unsubstituted or substituted aryl, unsubstituted or substituted heterocycle, unsubstituted or substituted C₃-C10 cycloalkyl, C₂-C₈ alkenyl, R¹⁰O—, or —N(R¹⁰)₂;
R² and R³ are independently selected from: H, unsubstituted or substituted C₁₋₆ alkyl, or 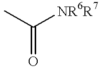
wherein the substituted group is substituted with one or more of:
1) aryl or heterocycle, unsubstituted or substituted with:
a) C₁-C₆ alkyl,
b) (CH₂)pOR⁶,
c) (CH₂)pNR⁶R⁷,
d) halogen,
e) CN,
2) C₃₋₆ cycloalkyl,
3) OR⁶,
4) SR^{6a}, S(O)R^{6a}, SO₂R^{6a}, 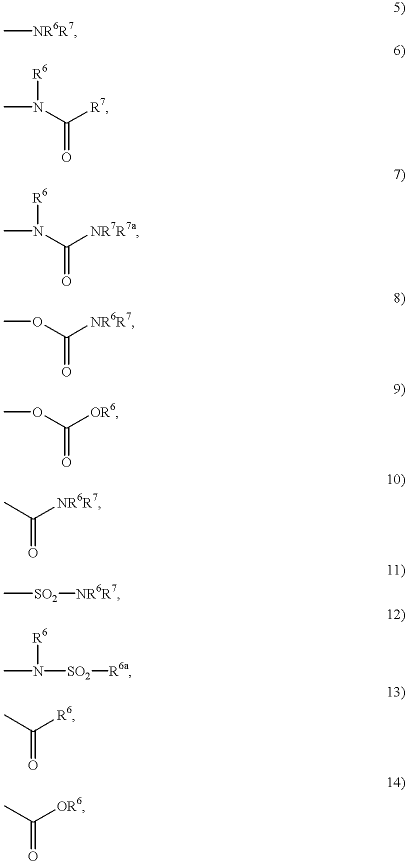
15) N₃, or
16) F; or
R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;
R⁴ is selected from H and unsubstituted or substituted C₁-C₆ alkyl;
and any two of R², R³ or R⁴ are optionally attached to the same carbon atom;
R⁵ is independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹ S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—,-N(R¹⁰)₂, or R¹10C(O)NR¹⁰—, and
c) C₁-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹⁰OC(O)NR¹⁰—;
R⁶, R⁷ and R^{7a} are independently selected from: H, C₁-C₆ alkyl, C₃₋₆ cycloalkyl, heterocycle, aryl, aroyl, heteroaroyl, arylsulfonyl, heteroarylsulfonyl, unsubstituted or substituted with:
a) C₁₋₆ alkoxy,
b) C₁-C₂₀ alkyl
c) aryl or heterocycle,
d) halogen,
e) HO,
f) —C(O)R¹¹,
g) —SO₂R¹¹, or
h) N(R¹⁰)₂; or
R⁶ and R⁷ may be joined in a ring;
R⁷ and R^{7a} may be joined in a ring;
R⁶a is selected from: C₁-C₆ alkyl, C₃₋₆ cycloalkyl, heterocycle, aryl, aroyl, heteroaroyl, arylsulfonyl, heteroarylsulfonyl, unsubstituted or substituted with:
a) C₁₋₆ alkoxy,
b) C₁-C₂₀ alkyl
c) aryl or heterocycle,
d) halogen,
e) HO,
f) —C(O)R¹
g) —S0₂R¹¹, or
h) N(R¹⁰)₂; or
R⁸ is independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹ S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and
c) C 1-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;
R⁹ is selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, R¹⁰⁾₂N—C(NR¹⁰)—, CN, NO₂, R¹⁰C(O)—, R¹⁰OC(O)—, N₃, —N(R¹⁰)₂, or R¹¹OC(O)NR¹⁰—, and
c) C₁-C₆ alkyl unsubstituted or substituted by aryl, heterocycle, C₃-C₁₀ cycloalkyl, perfluoroalkyl, halo, R10O—, R¹¹S(O)ₘ—, R¹⁰C(O)NR10—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;
R¹⁰ is independently selected from hydrogen, unsubstituted or substituted C₁-C₆ alkyl, perfluoroalkyl, unsubstituted or substituted aralkyl, and unsubstituted or substituted aryl;
R¹¹ is independently selected from unsubstituted or substituted C₁-C₆ alkyl and unsubstituted or substituted aryl;
A¹ and A² are independently selected from: a bond, —CH═CH—, —C≡C—, —C(O)—, —C(O)NR¹⁰—, —NR¹⁰C(O)—, O, —N(R¹⁰)—, —S(O)₂N(R¹⁰)—, —N(R¹⁰)S(O)₂—, or S(O)ₘ;
A³ is selected from —C(O)—, —C(R^{1a})₂—, O, —N(R¹⁰)— and S(O)ₘ;
W is a heterocycle selected from imidazolyl, pyridyl, thiazolyl, indolyl, quinolinyl, isoquinolinyl and thienyl;
Y is heteroaryl;
Z is a unsubstituted or substituted group selected from aryl, heteroaryl, arylmethyl, heteroarylmethyl, arylsulfonyl, heteroarylsulfonyl, wherein the substituted group is substituted with one or more of the following:
1) C₁-C₆ alkyl, unsubstituted or substituted with:
a) C₁₋₆ alkoxy,
b) NR⁶R⁷,
c) C₃₋₆ cycloalkyl,
d) aryl or heterocycle,
e) HO,
f) —S(O)ₘR^{6a}, or
g) —C(O)NR⁶R⁷,
2) unsubstituted or substituted aryl or unsubstituted or substituted heterocycle,
3) halogen,
4) OR⁶,
5) NR⁶R⁷,
6) CN,
7) NO₂,
8) CF₃;
9) —S(O)ₘR^{6a},
10) —C(O)NR⁶R⁷,
11) C₃-C₆ cycloalkyl,
12) —OCF₃, or
13) unsubstituted or substituted C₁₋₆ alkoxy;
m is 0, 1 or 2;
n is 0, 1, 2, 3 or 4;
p is 0, 1, 2, 3 or 4;
q is 0, 1 or 2;
r is 0 to 5;
t is 0 to 5;
u is 4 or 5; and
x is 0, 1, 2, 3 or 4;
or the pharmaceutically acceptable salts or optical isomers thereof.

3. The compound according to claim 1, as illustrated by formula C: 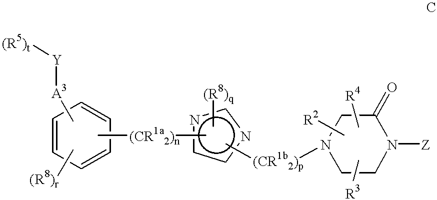
wherein:
R^{1a} and R^{1b} are independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, R¹OO—, or —N(R¹⁰)₂, or
c) unsubstituted or substituted C₁-C₆ alkyl wherein the substitutent on the substituted C₁-C₆ alkyl is selected from unsubstituted or substituted aryl, unsubstituted or substituted heterocycle, unsubstituted or substituted C₃-C₁₀ cycloalkyl, C₂-C₈ alkenyl, R¹OO—, or —N(R¹⁰)₂;
R² is H, unsubstituted or substituted C₁₋₆ alkyl, or 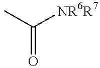
wherein the substituted group is substituted with one or more of:
1) aryl,
2) heterocycle,
3) OR⁶,
4) SR^{6a}, S0₂R^{6a}, or
5) 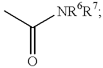
R³ and R⁴ are independently selected from H and unsubstituted or substituted C₁-C₆ alkyl;
and any two of R², R³ or R⁴ are optionally attached to the same carbon atom;
R⁵ is independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, Rl° C.(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and
c) C₁-C₆ alkyl unsubstituted or substituted by aryl, cyanophenyl, heterocycle, C₃-C₁₀ cycloalkyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;
R⁶ and R⁷ are independently selected from: H, C₁-C₆ alkyl, C₃₋₆ cycloalkyl, heterocycle, aryl, unsubstituted or substituted with:
a) C 1-6 alkoxy,
b) C₁-C₂₀ alkyl
c) aryl or heterocycle,
d) halogen, or
e) HO;
R⁶ and R⁷ may be joined in a ring; R⁶a is selected from: C₁-C₆ alkyl, C₃₋₆ cycloalkyl, heterocycle, aryl, aroyl, heteroaroyl, arylsulfonyl, heteroarylsulfonyl, unsubstituted or substituted with:
a) C₁₋₆ alkoxy,
b) C₁-C₂₀ alkyl
c) aryl or heterocycle,
d) halogen, or
e) HO;
R⁸ is independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰—, CN, NO₂,
R¹⁰C(O)—, R¹⁰OC(O)—, —N(R¹⁰)₂, or R¹¹OC(O)NR¹⁰—, and
c) C₁-C₆ alkyl unsubstituted or substituted by aryl, cyanophenyl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹⁰C(O)NR¹⁰-;
R⁹ is selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹⁰C(O)NR¹⁰—, and
c) C₁-C₆ alkyl unsubstituted or substituted by aryl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;
R¹⁰ is independently selected from hydrogen, unsubstituted or substituted C₁-C₆ alkyl, perfluoroalkyl, unsubstituted or substituted aralkyl, and unsubstituted or substituted aryl;
R¹¹ is independently selected from unsubstituted or substituted C₁-C₆ alkyl and unsubstituted or substituted aryl;
A³ is selected from —C(O)—, —C(R^{1a})₂—, O, —N(R¹⁰)— and S(O)ₘ;
Y is heteroaryl;
Z is a unsubstituted or substituted group selected from aryl, heteroaryl, arylmethyl, heteroarylmethyl, wherein the substituted group is substituted with one or more of the following:
1) C₁-C₆ alkyl, unsubstituted or substituted with:
a) C₁₋₆ alkoxy,
b) NR⁶R⁷,
c) C₃₋₆ cycloalkyl,
d) aryl or heterocycle,
e) HO,
f) —S(O)ₘR^{6a}, or
g) —C(O)NR⁶R⁷,
2) unsubstituted or substituted aryl or unsubstituted or substituted heterocycle,
3) halogen,
4) OR⁶,
5) NR⁶R⁷,
6) CN,
7) NO₂,
8) CF₃;
9) —S(O)ₘR^{6a},
10) —C(O)NR⁶R⁷,
11) C₃-C₆ cycloalkyl,
12) —OCF₃, or
13) unsubstituted or substituted C₁₋₆ alkoxy;
m is 0, 1 or 2;
n is 0, 1, 2, 3or 4;
p is 0, 1, 2, 3or 4;
q is 0, 1 or 2;
r is 0 to 5;
t is 0 to 5; and
u is 4 or 5;
or the pharmaceutically acceptable salts or optical isomers thereof.

4. A compound which is selected from: 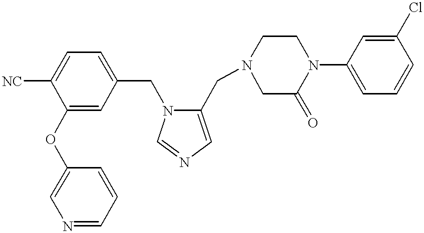
1-(3-chlorophenyl)-4-[1-(3-(3-pyridyloxy)-4-cyanobenzyl)-5-imidazolylmethyl]-2-piperazinone; 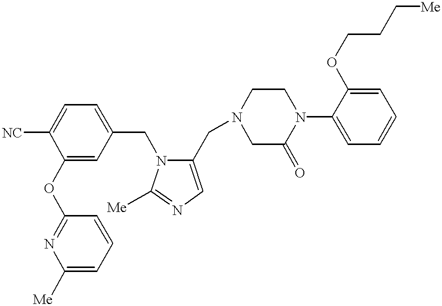
1-(2-(n-Butyloxy)phenyl)-4-[1-(3-((6-methyl-2-pyridyl)oxy)-4-cyanobenzyl)-2-methyl-5-imidazolylmethyl]-2-piperazinone;
or the pharmaceutically acceptable salts or optical isomers thereof.

5. A pharmaceutical composition comprising a pharmaceutical carrier, and dispersed therein, a therapeutically effective amount of a compound of claim 1.

6. A pharmaceutical composition comprising a pharmaceutical carrier, and dispersed therein, a therapeutically effective amount of a compound of claim 2.

7. A pharmaceutical composition comprising a pharmaceutical carrier, and dispersed therein, a therapeutically effective amount of a compound of claim 4.

8. A method for inhibiting farnesyl-protein transferase and geranylgeranyl-protein transferase type I which comprises administering to a mammal in need thereof a therapeutically effective amount of a compound of claim 1.

9. A method for inhibiting farnesyl-protein transferase and geranylgeranyl-protein transferase type I which comprises administering to a mammal in need thereof a therapeutically effective amount of a compound of claim 2.

10. A method for inhibiting farnesyl-protein transferase and geranylgeranyl-protein transferase type I which comprises administering to a mammal in need thereof a therapeutically effective amount of a compound of claim 4.

11. A method for treating cancer which comprises administering to a mammal in need thereof a therapeutically effective amount of a compound of claim 1.

12. A method according to claim 11 wherein the cancer is characterized by a mutated K4B-Ras p rotein.

13. A method for treating cancer which comprises administering to a mammal in need thereof a therapeutically effective amount of a compound claim 1.

14. A method for treating blindness related to retinal vascularization which comprises administering to a mammal in need thereof a therapeutically effective amount of a compound of claim 1.

15. A method for treating infections from hepatitis delta and related viruses which comprises administering to a mammal in need thereof a therapeutically effective amount of a compound of claim 1.

16. A method for preventing restenosis which comprises administering to a mammal in need thereof a therapeutically effective amount of a compound of claim 1.

17. A method for treating polycystic kidney disease which comprises administering to a mammal in need thereof a therapeutically effective amount of a compound of claim 1.

18. A pharmaceutical composition made by combining the compound of claim 1 and a pharmaceutically acceptable carrier.

19. A process for making a pharmaceutical composition comprising combining a compound of claim 1 and a pharmaceutically acceptable carrier.


SEQUENCE LISTING (21 sequences)

SEQ ID NO: 1 - protein, 4 aa, Homosapien
CVLS

SEQ ID NO: 2 - protein, 15 aa, Homosapien
GKKKKKKSKTKCVIM

SEQ ID NO: 3 - DNA, 52 bp, Artificial Sequence
  Completely synthesized
gagagggaattcgggcccttcctgcatgctgctgctgctgctgctgctgggc

SEQ ID NO: 4 - DNA, 41 bp, Artificial Sequence
  Completely synthesized
gagagagctcgaggttaacccgggtgcgcggcgtcggtggt

SEQ ID NO: 5 - DNA, 42 bp, Artificial Sequence
  Completely synthesized
gagagagtctagagttaacccgtggtccccgcgttgcttcct

SEQ ID NO: 6 - DNA, 43 bp, Artificial Sequence
  Completely synthesized
gaagaggaagcttggtaccgccactgggctgtaggtggtggct

SEQ ID NO: 7 - DNA, 27 bp, Artificial Sequence
  Completely synthesized
ggcagagctcgtttagtgaaccgtcag

SEQ ID NO: 8 - DNA, 27 bp, Artificial Sequence
  Completely synthesized
gagagatctcaaggacggtgactgcag

SEQ ID NO: 9 - DNA, 86 bp, Artificial Sequence
  Completely synthesized
tctcctcgaggccaccatggggagtagcaagagcaagcctaaggaccccagccagcgccggatgacagaatacaagcttgtggtgg

SEQ ID NO: 10 - DNA, 33 bp, Artificial Sequence
  Completely synthesized
cacatctagatcaggacagcacagacttgcagc

SEQ ID NO: 11 - DNA, 41 bp, Artificial Sequence
  Completely synthesized
tctcctcgaggccaccatgacagaatacaagcttgtggtgg

SEQ ID NO: 12 - DNA, 38 bp, Artificial Sequence
  Completely synthesized
cactctagactggtgtcagagcagcacacacttgcagc

SEQ ID NO: 13 - DNA, 38 bp, Artificial Sequence
  Completely synthesized
gagagaattcgccaccatgacggaatataagctggtgg

SEQ ID NO: 14 - DNA, 33 bp, Artificial Sequence
  Completely synthesized
gagagtcgacgcgtcaggagagcacacacttgc

SEQ ID NO: 15 - DNA, 22 bp, Artificial Sequence
  Completely synthesized
ccgccggcctggaggagtacag

SEQ ID NO: 16 - DNA, 38 bp, Artificial Sequence
  Completely synthesized
gagagaattcgccaccatgactgagtacaaactggtgg

SEQ ID NO: 17 - DNA, 32 bp, Artificial Sequence
  Completely synthesized
gagagtcgacttgttacatcaccacacatggc

SEQ ID NO: 18 - DNA, 21 bp, Artificial Sequence
  Completely synthesized
gttggagcagttggtgttggg

SEQ ID NO: 19 - DNA, 38 bp, Artificial Sequence
  Completely synthesized
gagaggtaccgccaccatgactgaatataaacttgtgg

SEQ ID NO: 20 - DNA, 36 bp, Artificial Sequence
  Completely synthesized
ctctgtcgacgtatttacataattacacactttgtc

SEQ ID NO: 21 - DNA, 24 bp, Artificial Sequence
  Completely synthesized
gtagttggagctgttggcgtaggc